(19) United States
(12) Patent Application Publication
(10) Pub. No.: US 2025/0375115 A1
(43) Pub. Date: Dec. 11, 2025

(54) SYSTEMS, DEVICES, AND METHODS FOR HEALTH MONITORING VIA IMPLANTABLE DEVICES IN THE ANTERIOR MEDIASTINUM

(71) Applicant: North American EP Technology LLC, Edina, MN (US)

(72) Inventors: Robert Joseph GASKILL, Edina, MN (US); Yanhui LI, Edina, MN (US)

(73) Assignee: North American EP Technology LLC, Edina, MN (US)

(21) Appl. No.: 19/300,150

(22) Filed: Aug. 14, 2025

Related U.S. Application Data
(63) Continuation of application No. PCT/US2025/014954, filed on Feb. 7, 2025.
(60) Provisional application No. 63/566,807, filed on Mar. 18, 2024.

(51) Int. Cl. A61B 5/0215; A61B 5/00

(52) U.S. Cl. CPC A61B 5/0215; A61B 5/686; A61B 5/725; A61B 5/7282

(57) ABSTRACT

A system includes a first sensor, a second sensor, and a sensing device coupled to the first sensor and the second sensor. The first sensor is disposed in an anterior mediastinum of a patient and is configured to detect a pressure signal therein. The second sensor is configured to detect a cardiac electrical signal. The sensing device includes a processor that is configured to execute instructions stored in a memory that cause the processor to (i) receive the pressure signal and the cardiac electrical signal, (ii) correlate the pressure signal and the cardiac electrical signal, (iii) determine, based on the correlated signals, at least one cardiac parameter, (iv) monitor the at least one cardiac parameter over a period of time to determine if a change in the cardiac parameter has occurred, and (v) responsive to determining the change has occurred, generate at least one action associated with the change.

### CROSS-REFERENCE TO RELATED APPLICATIONS

This application is a continuation of International Patent Application No. PCT/US2025/014954, filed Feb. 7, 2025, entitled “Systems, Devices, and Methods for Health Monitoring Via Implantable Devices in the Anterior Mediastinum,” which claims priority to and benefit of U.S. Provisional Patent Application No. 63/566,807, filed Mar. 18, 2024, entitled “Systems, Devices, and Methods for Improving Diagnostic Predictions using Multiple Data Sources,” the disclosure of each of which is incorporated herein by reference in its entirety.

This application is also related to International Patent Application No. PCT/US2024/042740, filed Aug. 16, 2024, entitled “Systems, Devices, and Methods for Improving Decision-Making of Implantable Devices Using Multiple Data Sources,” which claims priority to and the benefit of U.S. Provisional Patent Application No. 63/566,807, and which is a continuation-in-part of U.S. patent application Ser. No. 18/529,544, filed Dec. 5, 2023, entitled “Systems, Devices, and Methods for Improving Patient Outcomes in Implantable Cardioverter Defibrillators,” which claims priority to and the benefit of U.S. Provisional Patent Application No. 63/533,062, filed Aug. 16, 2023, entitled “Systems, Devices, and Methods for Improving Patient Outcomes in Implantable Cardioverter Defibrillators,” the disclosure of each of which is incorporated herein by reference in its entirety.

### BACKGROUND

The embodiments described herein relate generally to patient monitoring and more particularly, to systems, devices, and methods for improving patient monitoring and/or diagnostic predictions via implantable devices in the anterior mediastinum with measurements made without patient interaction.

Sensors are often used to measure certain characteristics associated with a patient. The characteristics and/or data indicative of or associated with the characteristics can be used for monitoring physiologic and/or pathophysiologic functions; diagnosing various diseases or disease states, health events, conditions, and/or injuries of a patient; and/or otherwise collecting health-related data for a patient. However, in some instances, sensors can generate signals that may lead to false positives due to incorrect, suboptimal, and/or less effective sensor placement, signal noise, and/or the like. The sensor signals may also lead to false positives if the sensor signal is associated with and/or indicative of an anomaly in a characteristic, but the anomaly is actually caused by and/or associated with something other than what is being diagnosed (i.e., a false positive).

For example, in some instances, a sensor may receive one or more cardiac electrical signals that may be associated with or indicative of arrhythmia, ventricular tachycardia, ventricular fibrillation, heart failure decompensation, COPD exacerbation, etc., but such signals may be influenced by other physiological and/or pathophysiological states. In some instances, such signals may accurately characterize an electrical state of a heart, but the state may resolve on its own after a relatively short time, or such an electrical state of the heart may not be associated with, may not coincide with, and/or may not cause an expected mechanical state of the heart (e.g., hemodynamic output). In such instances, relying single signal alone may lead to an inaccurate or incomplete understanding of the actual state of the heart and/or false positive diagnoses of a health event, condition, disease state, anomaly, etc. Thus, it may be desirable to collect data from and/or associated with one or more additional sources of bio-signals to verify and/or corroborate the data suggesting an anomaly in the characteristic.

In some instances, false positives (or false negatives) at the monitoring and/or diagnostic stage, in turn, can lead to false and/or inappropriate treatments being provided to a patient or inaccurate data presented to a physician. For example, false and/or inappropriate treatments may include providing unnecessary and/or incorrect medications, withholding a medication, inappropriate control of one or more implantable devices (e.g., a pacemaker, an implantable cardioverter defibrillator (ICD), a cardiac resynchronization therapy defibrillator (CRT-D), a ventricular assist device, a heart failure diagnostic device, a COPD diagnostic device, etc.). In the case of an ICD, for example, a false positive diagnosis of ventricular fibrillation may cause the ICD to provide inappropriate defibrillation shock treatment, which can be painful, potentially dangerous, and shown to increase all-cause mortality. False positives may also cause patients and/or physicians to lose confidence with a diagnostic/treatment system, which can lead to improper tuning and/or adjusting (e.g., to decrease sensitivity and/or otherwise reduce undesired treatments), or to non-use of the diagnostic/treatment system. In such instances, actual health events, conditions, and/or diseased states (i.e., true positives) may be missed, which can be dangerous or even deadly for a patient.

In addition, some known monitoring systems can be bulky and/or include sensors in or on multiple locations around the body. The monitoring systems are often used in and/or confined to a clinical or hospital setting but may not be suitable for use outside of such settings (e.g., are not ambulatory) and/or for use over an extended period of time. For example, some known systems for measuring pressure in portions of the chest cavity (e.g., the mediastinum) are non-ambulatory and may require hospitalization and/or partially implanted devices that can be restrictive to a patient's standard lifestyle. While some known ambulatory monitoring systems are implanted in the body, they are designed to measure a single parameter. Some such monitoring systems also require patients to initiate the measurement (e.g., a single point measurement, not continuous or at least semi-continuous measurements), which is dependent on patient compliance. Thus, there is a need for monitoring and/or diagnostic tool(s) that can be used by patients to make meaningful diagnoses or detect anomalies in patient characteristics and/or to monitor such characteristics over a period of time without patient interaction to make the measurement and/or that allows a patient to continue their standard lifestyle.

### SUMMARY

In some embodiments, a system includes a first sensor, a second sensor, and a sensing device coupled to the first sensor and the second sensor. The first sensor is disposed in an anterior mediastinum of a patient and is configured to detect a pressure signal therein. The second sensor is configured to detect a cardiac electrical signal. The sensing device includes a processor that is configured to execute instructions stored in a memory that cause the processor to (i) receive the pressure signal and the cardiac electrical signal, (ii) correlate the pressure signal and the cardiac electrical signal, (iii) determine, based on the correlated signals, at least one cardiac parameter, (iv) monitor the at least one cardiac parameter over a period of time to determine if a change in the cardiac parameter has occurred, and (v) responsive to determining the change has occurred, generate at least one action associated with the change.

In some embodiments, a non-transitory processor-readable medium stores code representing instructions that when executed by a processor, cause the processor to receive a cardiac electrical signal from an electrical sensor configured to detect electrical signals radiating from an external surface of a heart of a patient and a pressure signal from a pressure sensor disposed within an anterior mediastinum of the patient. The processor is caused to determine a diagnostic status based on the cardiac electrical signal. The processor is further caused to define a hemodynamic curve based at least in part on the pressure signal and to confirm the diagnostic status based at least in part on the hemodynamic curve. The processor is further caused to define a pulmonary curve based at least in part on the pressure signal and to confirm the diagnostic status based at least in part on the pulmonary curve. Responsive to confirming the diagnostic status, the processor is caused to generate at least one action associated with the diagnostic status.

In some embodiments, a method includes receiving pressure signals from at least one pressure sensor disposed within an anterior mediastinum of a patient and cardiac electrical signals from at least one electrode configured to receive cardiac electrical signals radiating from an external surface of a heart of the patient. A hemodynamic curve is defined based on the pressure signals and at least one cardiac parameter is determined based on the hemodynamic curve and the cardiac electrical signals. The method further includes monitoring the at least one cardiac parameter over a period of time to determine if a change in the cardiac parameter has occurred. In response to determining that the change in the cardiac parameter has occurred, the method includes generating at least one action associated with the change.

In some embodiments, a non-transitory processor-readable medium stores code representing instructions that when executed by a processor, cause the processor to receive pressure signals from at least one pressure sensor disposed within an anterior mediastinum of a patient and cardiac electrical signals from at least one electrode configured to detect electrical signals radiating from an external surface of a heart. The processor is caused to amplify and filter the pressure signals to separate the pressure signals into a plurality of frequency bands. The processor is caused to define a hemodynamic curve corresponding to the pressure signals in a first frequency band and a pulmonary curve corresponding to the pressure signals in a second frequency band different from the first frequency band. The processor is further caused to determine a change in a cardiac parameter over a period of time based on (i) the cardiac electrical signals, the hemodynamic curve, and the pulmonary curve and (ii) a change in at least one of the cardiac electrical signals, the hemodynamic curve, or the pulmonary curve over the period of time, and to generate at least one action based on the change in the cardiac parameter.

### BRIEF DESCRIPTION OF THE DRAWINGS

FIG. 1 is a schematic illustration depicting a sensing device including one or more sensors and engaging with a patient according to an embodiment.

FIG. 2A is an anterior view of a human thoracic cavity including various organs and structures, and depicting a placement of a sensing system in the anterior mediastinum according to an embodiment.

FIG. 2B is a sagittal cross-sectional view of the thoracic cavity of FIG. 2A, showing the different compartments or divisions of the mediastinum and surrounding structures, and depicting the sensing system of FIG. 2A in the anterior mediastinum according to an embodiment.

FIG. 3 schematically depicts a diagnostic/monitoring system engaging with a patient, according to an embodiment.

FIG. 4 is a flow chart depicting a method for determining if a patient is having a health event, according to an embodiment.

FIG. 5 is a flow chart depicting a method for determining a diagnostic status of a patient, according to an embodiment.

FIG. 6 is a schematic illustration of a sensing device with a lead, according to an embodiment.

FIG. 7 is a schematic illustration of a filtering process, according to an embodiment.

FIG. 8 is a flow chart depicting a method for determining at least one cardiac parameter, according to an embodiment.

FIG. 9 depicts a comparison between a hemodynamic output and an electrocardiogram signal, according to an embodiment.

FIG. 10 is a flow chart depicting a method for determining at least one pulmonary parameter, according to an embodiment.

FIG. 11 depicts a comparison between a respiratory output signal and an electrocardiogram signal, according to an embodiment.

FIG. 12 depicts a comparison between an electrocardiogram signal and a mediastinal pressure signal of normal sinus rhythm, according to an embodiment.

FIG. 13 depicts a comparison between an electrocardiogram signal and a mediastinal pressure signal during atrial fibrillation, according to an embodiment.

FIG. 14 depicts a comparison between an electrocardiogram signal and a mediastinal pressure signal during ventricular fibrillation conversion, according to an embodiment.

FIG. 15 depicts a comparison between an electrocardiogram signal and a mediastinal pressure signal during atrial fibrillatory to ventricular fibrillation, according to an embodiment.

FIG. 16 depicts a comparison between an electrocardiogram signal, a mediastinal pressure signal, and an atrial pressure signal, according to an embodiment.

FIG. 17 depicts a respiratory curve, according to an embodiment.

### DETAILED DESCRIPTION

The embodiments described herein relate generally to systems, devices, and/or methods for providing health-based monitoring of a patient and/or diagnostic predictions via implantable devices, for example, in the anterior mediastinum of the patient. In some embodiments, a diagnostic/monitoring system may include and/or be in communication with one or more sensors and/or other data sources to detect characteristics associated with the patient. The one or more characteristics can include one or more physiologic or pathophysiologic states as well as characteristics that are not detected by the sensors, such as patient demographic and/or health data (e.g., age, genetic information, health records, etc.). The one or more characteristics (or the underlying data indicative thereof) can be aggregated, correlated, verified, confirmed, corroborated, etc., which in turn, can improve diagnostic accuracy (e.g., reduce undesirable, inappropriate, and/or inaccurate diagnoses) by confirming that an indication of a health event, or the like, from one data source is occurring based on correlated data from other data sources. In addition or as an alternative, the diagnostic/monitoring system can be used to monitor the characteristic(s) to determine a diagnosis, identify an anomaly associated with the health of the patient, track a physiologic or pathophysiologic state of the patient over a period of time, and/or the like.

In some embodiments, the diagnostic/monitoring systems and/or methods described herein can be at least partially implemented in and/or can otherwise include an implantable cardiac treatment device (e.g., cardiac therapy device, defibrillator, implantable cardioverter defibrillator (ICD), cardiac resynchronization therapy defibrillator (CRT-D), pacemaker, etc.) configured to deliver treatment (shock therapy) based at least in part on one or more characteristics associated with a heart of a patient. Alternatively, the diagnostic/monitoring systems and/or methods described herein can be at least partially implemented in and/or can otherwise include an implantable diagnostic device configured to make diagnostic predictions and/or providing monitoring of one or more physiologic or pathophysiologic characteristics independent of whether a corresponding treatment is provided. It should be understood that the embodiments and methods described herein can be implemented as a diagnostic/monitoring system, a treatment system, a combined diagnostic/monitoring/treatment system, etc.

In some embodiments, a diagnostic/monitoring system (with or without treatment features) can include and/or can be in communication with one or more sensors configured to detect bio-signals associated with at least two physiologic or pathophysiologic characteristics. The characteristic(s) may be associated with the heart, the lungs, and/or any other suitable system of the body of the patient. For example, a first characteristic can be cardiac signals and/or one or more derivatives thereof, which can be determined based at least in part on data from one or more sensors (e.g., one or more sensors used for detecting cardiac electrical signals, referred to generally as an “ECG sensor”). As described above, using cardiac electrical signals alone to make diagnostic and/or treatment decisions can lead to false positives and/or undesirable, incorrect, and/or inappropriate detection, diagnostic, and/or therapy decisions. Accordingly, in at least one aspect, the devices, systems, and/or methods described herein can be configured to aggregate, combine, analyze, correlate, confirm, verify, and/or otherwise process the first characteristic, or data used to determine the first characteristic, with any other suitable characteristic(s) (or data) associated with the patient to determine, for example, if the heart is having an irregular and/or adverse event such as, for example, arrythmia, ventricular fibrillation, sudden cardiac arrest, etc. For example, such a process of correlating characteristics and/or data can include determining whether a cardiac rhythm seen in cardiac electrical signal data has an expected corresponding result in the hemodynamic status and/or output signal data. In such examples, if a potential irregular cardiac rhythm is detected without a corresponding irregular hemodynamic status and/or output a suspected or initially diagnosed cardiac state may not be confirmed or verified and/or a treatment such as shock therapy may not be delivered. Other examples include correlating the cardiac electrical signal with the hemodynamic signal to understand if a change has occurred in the heart failure status of a patient since the last measurement.

In some implementations, the other characteristic(s) and/or data can be, for example, hemodynamic status as measured by one or more sensors directly or indirectly. For example, one or more sensors can be a pressure sensor, transducer, etc. configured to measure changes in pressure. In some implementations, the sensor(s) can be configured to directly measure and/or detect a hemodynamic status or a pressure associated with the hemodynamic status (e.g., blood pressure, arterial pressure, stroke volume, etc.). In some implementations, the sensor(s) can be configured to indirectly measure and/or detect a hemodynamic status or a pressure associated with the hemodynamic status. For example, in some embodiments, a diagnostic/monitoring system (with or without treatment features) can include and/or can be in communication with one or more pressure sensors, transducers, and/or the like (referred to generally as “pressure sensor”), which is/are disposed in a substernal space (or anterior mediastinum) of the patient and in contact with and/or in close proximity to the free wall of either the right, left, or both ventricles. In such embodiments, movement associated with the pumping/beating of the heart can result in pressure changes in the tissue or volumes surrounding the heart, which in turn, can be measured and/or detected by the pressure sensor. The changes in pressure are inverse to the changes in pressure in the heart as a contraction of the heart would decrease pressure in the anterior mediastinum (e.g., increase of volume in the anterior mediastinum) while increasing pressure within the heart (e.g., to pump blood out of the heart). The one or more pressure sensors can similarly be configured to detect, sense, measure, etc. pressure changes associated with the movement and/or functioning of the lungs (i.e., respiration), a background mediastinal pressure, and/or the like. Similar to the heart, the changes in pressure in the anterior mediastinum can be inverse to the pressure changes in the pulmonary system, or more specifically, the lungs.

In some implementations, inputs from the pressure sensor (and/or any other sensor) can be used and/or correlated with inputs of the ECG sensor to detect and/or determine a cardiac status and/or the occurrence of health events, such as arrhythmia, tachycardia, bradycardia, specific pressure measurements associated with hemodynamics, respiratory signals, and/or the like. The cardiac status determined using the methods described herein can be more specific than determining cardiac status using cardiac electrical signal measurements alone, hemodynamic status measurements alone, and/or other cardiac characteristics individually. Using a combination of cardiac signal measurements such as cardiac electrical signal measurements, cardiac mechanical signal measurements (e.g., hemodynamic rate, status, and/or output measurements), and/or other bio-signal measurements (e.g., pressure changes in the substernal space) can increase sensitivity and specificity, thereby reducing false results (false positives and false negatives). Additionally, detecting, sensing, and/or determining hemodynamic status can further confirm arrythmias, ventricular fibrillation, atrial fibrillation, ventricular tachycardias, heart failure status, COPD status, and/or other cardiac states. This results in specificity in diagnostic decisions that is more beneficial to patients and supported by clinical evidence. In implementations in which the system includes a treatment device, cardiac pacing and/or shock treatment decisions can be made based on the signals from the one or more sensors (e.g., electrical signal measurements such as ECG signal measurements, mechanical signal measurements such as hemodynamic signal measurements, and/or any other bio-signal measurements such as respiratory signal measurements and/or the like) and/or the correlated data associated therewith (e.g., ex vivo data such as patient demographic and/or health data-age, weight, cardiac pathology, genetic information, health records, etc.).

In some embodiments, the diagnostic/monitoring system may include one or more sensing devices implanted in a patient to detect and/or sense bio-signals associated with one or more characteristic. In some embodiments, the sensing device(s) may be and/or may be used in conjunction with a wearable (e.g., smart watch, wrist cuff, ankle cuff, chest strap, smart ring, etc.). In some embodiments, the sensing device(s) may be configured to process, aggregate, correlate, and/or the like, data or may be configured to send data to a compute device for processing. In some embodiments, the sensing device can be configured to detect signals within or associated with the anterior mediastinum. In some embodiments, the sensing device can be configured to detect cardiac electrical signals, such as an electrocardiogram (ECG) signal associated with the heart of the patient. In some embodiments, the sensing device can be configured to measure one or more pressure signal(s) within or associated with the anterior mediastinum. The pressure signal can be affected by both the changes in heart volume and the changes in lung volume during the cardiac cycle and respiratory cycle, respectively as the thoracic cavity is a closed chamber which includes the lungs and the heart.

In some embodiments, the data from the sensing device(s) can be processed, aggregated, correlated, and/or filtered to determine the one or more physiologic or pathophysiologic characteristics, states, etc. In some embodiments, the data can be amplified and filtered. For example, the pressure data can be amplified and filtered to separate the pressure signal data into different frequency bands. A set of pressure curves is generated based on the separated pressure signals in the different frequency bands. Similarly, the cardiac electrical signals can be amplified and filtered. Cardiac curves (also referred to as “hemodynamic curves”) can also be generated. In some embodiments, the pressure curve can be correlated with the cardiac curves. In some embodiments, the one or more of the pressure curves can be used to determine a physiological status of the patient.

In some embodiments, the data from the sensing device(s) can include an electrical sensor and at least one pressure sensor that are configured to be disposed within a mediastinal space of the patient. The electrical sensor is configured to detect electrical signals radiating from a heart of the patient. An amplifier/filter is coupled to the at least one pressure sensor and is configured to amplify and filter pressure signal data received from the at least one pressure sensor into pressure signals in different frequency bands. A compute device (or a processor thereof) can (i) receive the pressure signals in the different frequency bands, (ii) generate at least one pressure curve based on the pressure signals in different frequency bands, and (iii) receive, from the electrical sensor, data associated with the electrical signals radiating from the heart. The data associated with the at least one pressure curve can be correlated with the data associated with the electrical signals, which can allow the diagnostic/monitoring system to determine a physiological status of the patient based on the correlation.

In some embodiments, a diagnostic/monitoring system can be configured to use the data received from one or more sensing device(s) to provide one or more diagnoses, determine if a medical event is occurring, and/or determine if an anomaly is present. For example, the characteristics and/or the physiological status can be used. In some embodiments, the diagnostic/monitoring system can be configured to monitor the characteristics associated with the patient over a period of time. In some embodiments, the monitoring system is configured to generate and/or trigger a notification and/or alarm system. The notification system can be configured to notify a user, a patient, a medical professional, an emergency responder, and/or the like of the diagnoses, changes in measurements, trends in measurements, medical event(s), and/or anomaly and associated information. In some embodiments, the notification system can include and/or can be configured to provide one or more signals to an external and/or remote device such as a smartphone, a smart watch, and/or the like, which in turn, may provide a notification to the user (e.g., via a mobile application, etc.). In some embodiments, the notification system can include and/or can be configured to provide one or more signals that trigger an alarm (e.g., in a hospital or medical facility setting), place an emergency (911) call, etc.

In some embodiments, one or more machine learning models can be used for processing the data from the sensing device and providing, as an output, a predictive diagnosis. In some embodiments, various machine learning models can be used for processing data from any number of data sources, where each machine learning model is specifically trained to predict a health state/event based on a set or type of data (e.g., making a prediction based on cardiac electrical data, making a prediction based on cardiac mechanical data, making a prediction based on respiratory data, and/or the like). A different machine learning model can then be used for aggregation and/or correlation of the data and/or predictions from each machine learning model to determine (or at least corroborate) if a health event is occurring or for diagnosis.

The terminology used herein is for the purpose of describing particular embodiments, implementations, and/or concepts (including any feature(s) or aspect(s) thereof) and is not intended to be limiting. Unless defined otherwise, technical and/or scientific terms used herein have the same meanings as commonly understood by one of ordinary skill in the art. Any explanation or discussion of or using particular terms is intended to provide context and to facilitate understanding and is not necessarily intended to replace or supersede commonly used or known definitions understood by one skilled in the art unless explicitly stated otherwise. Moreover, various terms may be used to describe similar or substantially the same embodiments, implementations, and/or concepts (including any feature(s) or aspect(s) thereof) and thus, the use of particular term is not intended to be limiting and/or to the exclusion of other terms unless the terms are mutually exclusive, or the context clearly states otherwise.

As used herein, the singular forms “a,” “an,” and “the” are intended to include the plural forms as well, unless the context clearly indicates otherwise. With respect to the use of singular and/or plural terms herein, those having skill in the art can translate from the singular to the plurality and/or vice versa as is appropriate for the context and/or application. Furthermore, any reference herein to a singular component, feature, aspect, etc. is not intended to imply the exclusion of more than one such component, feature, aspect, etc. (and/or vice versa) unless expressly stated otherwise. The various singular/plural permutations may be expressly set forth herein for sake of clarity.

In general, terms used herein and in the appended claims are intended as “open” terms unless expressly stated otherwise. For example, the term “including” should be interpreted as “including but not limited to,” the term “having” should be interpreted as “having at least,” etc. Similarly, the term “comprising” may specify the presence of stated features, elements, components, integers (or fractions thereof), steps, operations, and/or the like but does not preclude the presence or addition of one or more other features, elements, components, integers (or fractions thereof), steps, operations, elements, components, and/or groups thereof, and/or the like unless such combinations are otherwise mutually exclusive.

As used herein the term “and/or” includes any and all combinations of one or more of the associated listed items. It should be understood that any suitable disjunctive word and/or phrase presenting two or more alternative terms, whether in the written description or claims, contemplates the possibilities of including one of the terms, either of the terms, or both/all of the terms. For example, the phrase “A and/or B” will be understood to include the possibilities of “A” alone, “B” alone, or a combination of “A and B.”

All ranges described herein include each individual member or value and are intended to encompass any and all possible subranges and/or combinations of subranges thereof unless expressly stated otherwise. Any listed range should be recognized as sufficiently describing and enabling the same range being broken down into at least equal subparts unless expressly stated otherwise.

As used herein, the terms “about,” “approximately,” and/or “substantially” when used in connection with stated value(s) and/or geometric structure(s) or relationship(s) is intended to convey that the value or characteristic so defined is nominally the value stated or characteristic described. In some instances, the terms “about,” “approximately,” and/or “substantially” can generally mean and/or can generally contemplate a value or characteristic stated within a desirable tolerance (e.g., plus or minus 10% of the value or characteristic stated). For example, a value of about 0.01 can include 0.009 and 0.011, a value of about 0.5 can include 0.45 and 0.55, a value of about 10 can include 9 to 11, and a value of about 1000 can include 900 to 1100. Similarly, a first surface may be described as being substantially parallel to a second surface when the surfaces are nominally parallel. While a value, structure, and/or relationship stated may be desirable, it should be understood that some variance may occur as a result of, for example, manufacturing tolerances or other practical considerations (such as, for example, the pressure or force applied through a portion of a device, conduit, lumen, etc.). Accordingly, the terms “about,” “approximately,” and/or “substantially” can be used herein to account for such tolerances and/or considerations.

As used herein, the words “proximal” and “distal” refer to a direction and/or position relative to a reference. The words “proximal” or “distal” can be relative terms and do not necessarily refer to universally fixed directions or positions. For example, in the context of a device that is manipulated by a user to engage a body of a patient, the terms “proximal” and “distal” generally refer to a direction and/or position that is closer to and away from, respectively, the user who would place the device into contact or engagement with the patient. Similarly state, an end or end portion of a device first touching the body of the patient would be the distal end or distal end portion, while the opposite end or end portion of the device (e.g., the end or end portion of the device being manipulated by the user) would be the proximal end or proximal end portion of the device. In the context of a device implanted in the body of a patient, an end or end portion of the device that is closer to the heart of the patient would be the distal end or distal end portion, while the opposite end or end portion (e.g., the end or end portion further from the heart) would be the proximal end or proximal end portion of the device.

As used herein, the term “characteristic(s)” described in reference to a patient generally refers to information associated with physiological and/or pathophysiological bio-signals of the patient. Such signals can be, for example, electrical signals, non-electrical (e.g., mechanical) signals, temperature-related signals, chemical or composition-related signals, and/or the like. Electrical signals can include any suitable signals associated with and/or otherwise indicative of the electrical functioning of the heart. The embodiments and/or methods described herein generally include implanting leads and/or sensors thereof in the anterior mediastinum of the patient, which can detect such electrical signals radiating from the heart. The detection and/or measurement of such cardiac electrical signals may include, but is not limited to, heart rate, voltage, P wave, QRS morphology, ST segment, T wave, ECG diagnosis, and/or the like, sensed through any suitable number of vectors. In some implementations, the characteristics can include, but are not limited to, cardiac or non-cardiac information and/or bio-measurements such as cardiac electrical signals, cardiac mechanical signals, and/or signals associated with cardiac cycle, pulmonary cycle, nervous system, body temperature, glucose level, pressure characteristics (e.g., blood pressure, pressure in the tissue or volumes surrounding the heart such as in the mediastinum, venous pressures, arterial pressures, and/or changes in such pressures, etc.), hemodynamic characteristics, volumetric characteristics (e.g., blood volume in the circulation system and/or changes thereof such as those detected via photoplethysmography (PPG)), oxygen saturation, sensed mechanical heart movement, cardiac sounds, cardiac echogram (ultrasound), cardiac Doppler, sleep performance, sleep apnea, recovery status, hemodynamic status, activity level, heart rate, respiratory rate, heart failure, and/or the like), data from internal or implanted medical devices (e.g., a pacemaker, an ICD, a CRT-D, ventricular assist device (VAD), a prosthetic device such as a heart valve prosthesis, etc.), and/or the like.

The embodiments described herein and/or portions thereof can be formed or constructed of one or more biocompatible materials. In some embodiments, the biocompatible materials can be selected based on one or more properties of the constituent material such as, for example, stiffness, toughness, durometer, bioreactivity, etc. Examples of suitable biocompatible materials include but are not necessarily limited to metals, glasses, ceramics, and/or polymers. Examples of suitable metals include pharmaceutical grade stainless steel, gold, titanium, nickel, iron, platinum, tin, chromium, copper, and/or alloys thereof. A polymer material may be biodegradable or non-biodegradable. Examples of suitable biodegradable polymers include polylactides, polyglycolides, polylactide-co-glycolides, polyanhydrides, polyorthoesters, polyetheresters, polycaprolactones, polyesteramides, poly(butyric acid), poly(valeric acid), polyurethanes, biodegradable polyamides (nylons), and/or blends and copolymers thereof. Examples of non-biodegradable polymers include non-degradable polyamides (nylons), polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, and/or blends and copolymers thereof.

Non-limiting examples of suitable biocompatible polymer materials can include polylactides, polyglycolides, polylactide-co-glycolides, polyethylene-glycols, polyanhydrides, polyorthoesters, polyetheresters, polycaprolactones, polyesteramides, poly(butyric acid), poly(valeric acid), polyurethanes, polyamides (nylons), polyesters, polycarbonates, polyacrylates, polystyrenes, polypropylenes, polyethylenes, polyethylene oxide, polyolefins, polyethersulphones, polysulphones, polyvinylpyrrolidones, polyvinyl chloride, polyvinyl fluoride, poly(vinyl imidazole), polyether urethanes, silicone polyether urethanes, polyetheretherketones (PEEK), polytetrafluoroethylenes (PTFE), polylactones, chlorosulphonate polyolefins, ethylene-vinyl acetates and other acyl substituted cellulose acetates, elastomers, thermoplastics, and/or blends and copolymers thereof.

The embodiments, methods, and/or implementations herein, and/or the various features or advantageous details thereof, are explained more fully with reference to the non-limiting examples illustrated in the accompanying drawings and detailed in the following description. The examples and/or embodiments described herein are intended to facilitate an understanding of structures, functions, and/or aspects of the embodiments, ways in which the embodiments may be practiced, and/or to further enable those skilled in the art to practice the embodiments herein. Similarly, methods and/or ways of using or implementing the embodiments described herein are provided by way of example only and not limitation. Specific uses and/or implementations described herein are not provided to the exclusion of other uses unless the context expressly states otherwise. Descriptions of well-known components, methods, techniques, etc. may be omitted so as to not obscure the embodiments herein. Like numbers refer to like elements throughout.

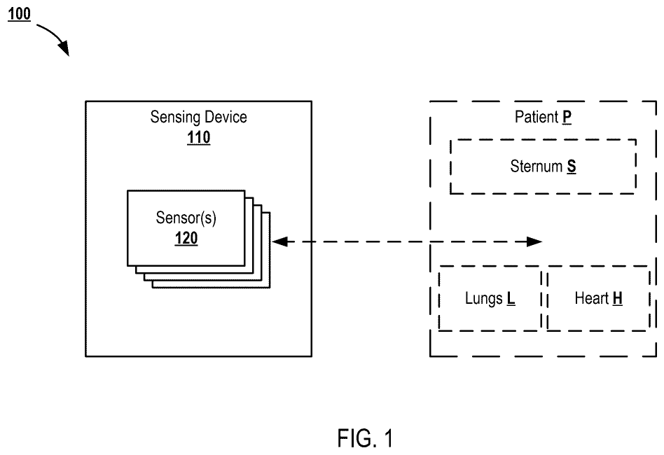

FIG. 1 is a schematic illustration depicting a diagnostic/monitoring system 100 engaging a patient P according to an embodiment. The diagnostic/monitoring system 100 (referred to herein as “system 100”) includes a sensing device 110 having and/or in communication with a set of sensor(s) 120. In some embodiments, the sensing device 110 is configured to be implanted in the patient P. For example, at least one sensor of the sensor(s) 120 of the sensing device 110 is placed or implanted between a heart H and/or the lungs L and a sternum S of the patient P, as depicted.

In some implementations, the sensing device 110 can be permanently implanted or temporarily placed between the heart H and the sternum S. For example, the sensing device 110 can be implanted in the anterior mediastinum. The sensing device 110 can be any suitable device configured to perform any number of diagnostic, monitoring, and/or sensing processes based at least in part on data received from the set of sensor(s) 120. In some embodiments, for example, the sensing device 110 can be configured to analyze and/or process data (including, but not limited to, data from the set of sensor(s) 120) during monitoring to determine one or more diagnosis, health event, changes in measurements over time, trends in measurements, and/or anomaly associated with the health of the patient P. The determinations and/or the detected characteristics of the sensing device 110 can be associated with, for example, the health and/or functioning of the patient's heart, lungs, and/or other portions of the patient's body. In some embodiments, the sensing device 110 may be an ambulatory device that can allow for a patient P to walk and/or complete other daily activities while having the sensing device 110 implanted in the body. For example, the sensing device 110 can be used for ambulatory monitoring that can provide monitoring of the patient P outside of a clinical and/or hospital setting. The sensing device 110 may be referred to as an “ambulatory device” as it may be implanted using minimally invasive procedures and function so as to not obstructively impact the life of the patient P.

In some embodiments, the sensing device 110 can be implemented in or as a treatment and/or therapy device such as an implantable cardioverter-defibrillator (ICD), a cardiac resynchronization therapy defibrillator (CRT-D), a pacemaker, and/or any other suitable device. For example, the sensing device 110 can be configured to analyze and/or process data (including, but not limited to, data from the sensor(s) 120) to inform and/or to make one or more decisions associated with providing treatment and/or therapy to the patient P. For example, in the case of an ICD, the sensing device 110 can be configured to determine whether to provide electric shock therapy (e.g., a defibrillation shock, cardiac pacing, and/or the like) to the heart H of the patient P based at least in part on data received from the set of sensor(s) 120. In some embodiments, the sensing device 110 can be configured to provide and/or perform diagnostic and/or monitoring as well as treatment functionality. Specifically, when the sensing device 110 is implemented in or as a treatment and/or therapy device, the sensing device 110 can be configured to detect a trigger (e.g., health event, etc.) as to inform or confirm an urgent treatment decision. When implemented to provide and/or perform diagnostic and/or monitoring, the sensing device 110 can monitor health characteristics associated with the patient over a period of time to determine if the dynamics (e.g., changes, etc.) of the health characteristics indicate an anomalous trend that may be indicative of a pathology, disease, congenital defect, anatomical defect, and/or the like.

The set of sensor(s) 120 can include any number of sensors configured to detect bio-signals and/or other signals associated with a patient. For example, the set of sensor(s) 120 can include one or more sensors implanted in the body and configured to detect and/or measure one or more characteristics and/or signals associated with the cardiovascular system, the respiratory system, and/or any other suitable system or portion of the body. In some embodiments, one or more sensors can be disposed outside of the body (e.g., included in a wearable such as a smartwatch, fitness tracker, an insulin pump, a thermometer, a pulse oximeter, a smart ring, and/or the like). In some embodiments, the set of sensor(s) 120 can include a cardiac electrical sensor configured to measure intracardiac signals of the heart H such as electrocardiogram (ECG) signals and/or electrogram signals. In some embodiments, the sensor(s) 120 can include sensors external to the patient P configured to measure cardiac electrical signals. The set of sensor(s) 120 can include one or more pressure sensors. For example, the pressure sensor can be configured to measure the pressure within the anterior mediastinum. In some embodiments, the set of sensor(s) 120 can include additional sensors such a photoplethysmography sensor (PPG) sensor (or other optical sensor), an oxygen saturation (SpO2) sensor, an accelerometer, a temperature sensor, an acoustic sensor, an ultrasound sensor, an optical sensor, and/or the like. In some embodiments, the sensor(s) 120 can be configured to monitor and/or measure multiple characteristics associated with the patient P, which in turn, can be used to determine and/or define one or more treatment decisions, diagnoses, diagnostic predictions, anomalies, health events and/or the like. As described in detail herein, the data associated with and/or indicative of the multiple characteristics and sources can be correlated, aggregated, confirmed, verified, etc. to allow for more accurate and precise diagnostic and/or treatment decisions than a diagnostic and/or treatment decision using just one characteristic.

In some embodiments, a first sensor can be configured to detect and/or measure at least one characteristic of or associated with the heart H and a second sensor configured to detect and/or measure at least one characteristic within the anterior mediastinum that is different from the characteristic(s) measured by the first sensor. The second sensor may be configured to detect and/or measure a pressure in the space (e.g., portion of the body) in which the second sensor is placed (e.g., in the anterior mediastinum). The sensing device 110 with the set of sensors may be configured to monitor, diagnose, and/or treat a patient's health (e.g., the sensing device 110 may be a diagnostic device only, a monitoring device only, a therapeutic device only, or any suitable combination of diagnostic device (or function), monitoring device (or function), and therapeutic device (or function)).

In some embodiments, the coordination of the data obtained from the set of sensor(s) 120 can be used to determine and/or monitor patient health information. This can be performed, for example, by selectively separating the data received from one or more sensor(s) 120 as a function of the source, characteristic, and/or bio-signal being detected. For example, any suitable amplification and filtering (either digitally or through physical circuitry) can be performed on raw sensor data to separate the data into multiple signals, vectors, modalities, characteristics, etc. In some embodiments, the sensor(s) 120 can include one or more pressure sensors that can sense and/or detect pressures and/or pressure changes in, for example, the anterior mediastinum. In such embodiments, the data can be separated based on the physiological and/or pathophysiological characteristic producing the pressure signal. For example, the pressure data can be separated (e.g., via amplification and/or filtering) into a respiratory pressure curve, a cardiac pressure curve, various cardiac pressures associated derived from the pressure measurements, and a mediastinal pressure curve. In some embodiments, the amplification and/or filtering can be performed based at least in part on differing frequencies within the pressure data as described in detail below with reference to specific embodiments. Moreover, understanding the individual pressure curves associated with the physiologic and/or pathophysiologic cause of the pressure changes can allow for improved monitoring (and/or improved specificity of the collected or measured data), which in turn, can result in meaningful health alerts and/or decisions (with reduced false positives and/or false negatives).

In some embodiments, the sensing device 110 can include one or more lead configured to access various portions of the mediastinal space between the heart H and the sternum S. For example, the anterior mediastinal space is a volume in the thoracic cavity between the right lung and the left lung (lungs L) and between the heart H and a posterior surface of the sternum S. As such, the functioning and/or changes in the functioning of the heart H and lungs L affect the pressure within the anterior mediastinum. The lead can be configured to deliver the sensor(s) 120 to a desired location. For example, the lead can be configured to position at least one of the sensor(s) 120 against the heart H to measure cardiac electrical signals and/or any other signals within the anterior mediastinum.

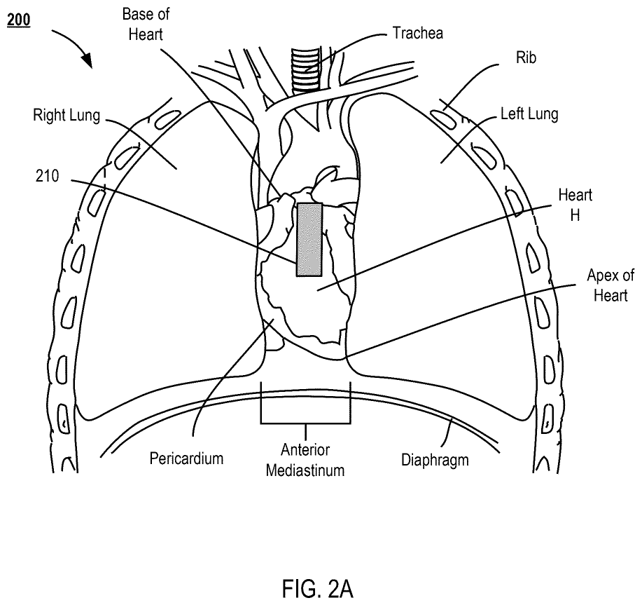

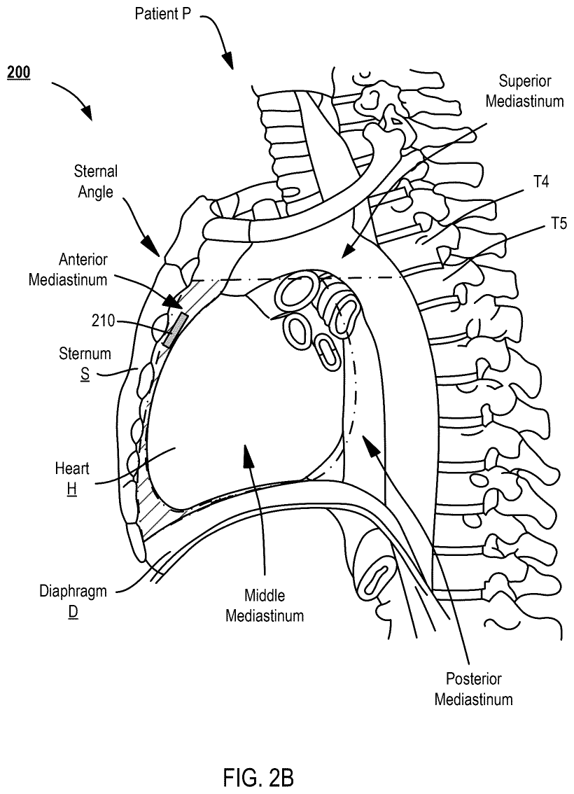

FIGS. 2A and 2B are different views of the human thoracic cavity and are shown to provide reference and context for the discussion of the various embodiments described herein. Specifically, FIG. 2A is an anterior view of the human thoracic cavity which illustrates various organs and structures. FIG. 2A depicts a sensing device 210 (e.g., functionally and/or structurally similar to the sensing device 110 of FIG. 1) implanted in the thoracic cavity forward or anterior to the heart. The sensing device 210 is a portion of a diagnostic/monitoring system 200 (e.g., functionally and/or structurally similar to the diagnostic/monitoring system 100 of FIG. 1). In some embodiments, only a portion of the sensing device 210 is disposed in the thoracic cavity. For example, a lead of the sensing device 210 may be positioned in the chest while the remainder of the sensing device 210 is located elsewhere in the body of the patient P and/or outside of the body.

FIG. 2B is a sagittal cross-sectional view of the thoracic cavity of FIG. 2A, showing the different compartments or divisions of the mediastinum and surrounding structures. T4 and T5 are shown, which are the fourth and fifth thoracic vertebrae, respectively, that form a portion of the patient's spine. A sternal angle is shown, which is a joint that serves as a landmark to locate a second rib of a ribcage and a level of an intervertebral disc between T4 and T5. A superior mediastinum is shown, which is a region that extends from a top of the thoracic cavity down to the sternal angle and includes structures like a trachea, esophagus, and major blood vessels. The anterior mediastinum is shown between the sternum S (the bones below the sternal angle) and the heart H, superior to the diaphragm. A middle mediastinum is located centrally in the thoracic cavity and includes the heart H and roots of main blood vessels (e.g., aortic artery, pulmonary vein, etc.). A posterior mediastinum is also shown, which is behind the heart H and in front of the spine.

As seen in FIG. 2B, the sensing device 210 is positioned in the anterior mediastinum corresponding to the substernal space between the sternum S and the heart H of the patient P. In some embodiments, at least a portion of the sensing device 210 may be in contact with a portion of the heart H. The positioning of the sensing device 210 can be configured to allow for detecting or one or more desired bio-signal. For example, the sensing device 210 can be positioned in the anterior mediastinum to allow for detecting a pressure associated with the anterior mediastinum and cardiac electrical signals associated with the heart H. The sensing device 210 is configured to facilitate and/or allow the natural movement of the heart H that occurs with each cardiac cycle. In some embodiments, the sensing device 210 can facilitate and/or allow the natural movement by using the sternum S as a base and having the sensing device 210 pressed against or otherwise placed in contact with the fibrous layer of the pericardium. This position allows for the sensing device 210 to move in multiple directions and absorb and/or move with the motion of the heart H. This position also allows the sensing device 210 to detect pressure changes in the anterior mediastinum associated with the pulmonary system (e.g., expansion of the lungs during inhalation and the contraction of the lungs during exhalation).

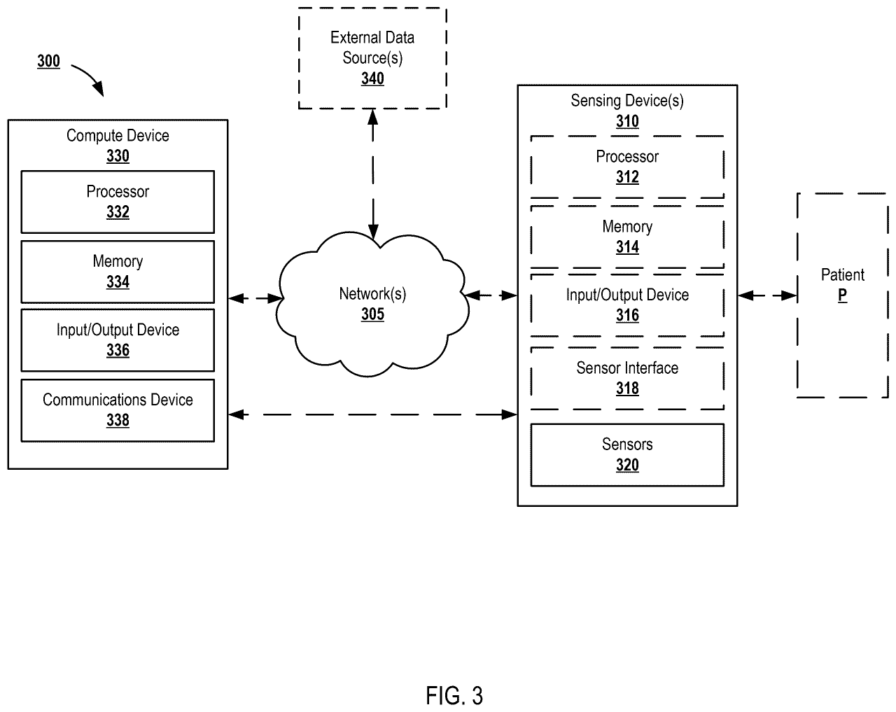

FIG. 3 schematically depicts a diagnostic/monitoring system 300 engaging a patient P, according to an embodiment. In some embodiments, the diagnostic/monitoring system 300 (“system 300”) can be similar to and/or can be a specific implementation of the system 100 described above with reference to FIG. 1. The diagnostic/monitoring system 300 can be utilized for monitoring a patient, determining if a health event is occurring, and/or diagnosing health conditions and/or characteristics of a patient. As described herein, the system 300 uses data from one or more sensing device(s) for monitoring the health of a patient P and/or making diagnoses or diagnostic predictions. In some embodiments, the system 300 can be used to make diagnostic predictions and/or the like based at least in part on data from multiple data sources, which in turn, can decrease the likelihood of a false positive diagnosis and/or delivery or an undesired or inappropriate therapy or treatment. Although the system 300 is described herein with reference to FIG. 3 as being used to monitor and/or diagnose (or make diagnostic predictions based on data associated with) a patient, in some embodiments, the system 300 can be used to make therapeutic and/or treatment decisions and/or otherwise deliver one or more therapies or treatments that may, for example, correspond to and/or treat a disease state or health condition that is being monitored by or that is diagnosed and/or predicted by one or more sensing device(s).

As shown, the system 300 includes sensing device(s) 310 (e.g., structurally and/or functionally similar to the sensing device 110 of FIG. 1 and/or the sensing device 210 of FIGS. 2A and 2B) engaging and/or implanted in a patient P. The system 300 further includes a compute device 330 and one or more optional external data source(s) 340. The sensing device(s) 310, the compute device 330, and/or the external data source(s) 340 may be communicably coupled via network(s) 305. The system 300 uses at least the sensing device(s) 310 and the compute device 330, and optionally the external data source(s) 340 (and/or data from these or other devices) for monitoring the health of the patient P. Any of the components, devices, and/or aspects of the system 300 can be similar in at least form and/or function to corresponding components, devices, and/or aspects of the system 100 described above with reference to FIG. 1. Accordingly, some such components, devices, and/or aspects of the system 300 may not be described in further detail herein and should be considered as structurally and/or functionally similar to the corresponding components, devices, and/or aspects of the system 100 unless stated otherwise. A brief discussion of the structural, electrical, and/or electronic components of the system 300 is provided below followed by a discussion of implementations, methods, and/or examples of using the system 300 (or portions thereof) to monitor the health of the patient P, to generate one or more diagnoses and/or diagnostic predictions, and/or to perform any other suitable health-related functions.

The network(s) 305 shown in FIG. 3 can be and/or can include one or more network(s) that may be any type of network or combination of networks (e.g., a local area network (LAN), a wide area network (WAN), a metropolitan area network (MAN), a worldwide interoperability for microwave access network (WiMAX), a virtual network (e.g., VLAN), an intranet, the Internet, an optical fiber (or fiber optic)-based network, a telecommunication network, a cellular network, and/or any other suitable network or combinations thereof). The network can be implemented as a wired network and/or wireless network (e.g., via Wi-Fi®, Bluetooth®, Bluetooth® low energy, Zigbee, Z-Wave, near field communication (NFC), Thread, Matter, etc.). Accordingly, the network(s) 305 can be used to operatively couple any number of compute devices (or other electric or electronic devices) including, for example, the compute device 330, the external data source(s) 340, and/or the sensing device(s) 310.

The compute device 330 shown in FIG. 3 is and/or includes one or more external devices (e.g., external to the sensing device(s) 310 and/or the patient P) that is in communication with the external data source(s) 340 and the sensing device(s) 310 via the network(s) 305. The compute device 330 may be any suitable device or combination of devices configured to send, receive, process, analyze, store, use, change, define, etc. data, data structures, and/or the like. Moreover, the compute device 330 can be configured to perform one or more processes, functions, applications, programs, signal processing, algorithms, models, etc. The components of the compute device 330 can be contained within a single housing or machine or can be distributed within and/or between multiple physical machines, virtual machines, and/or any combination thereof. In some embodiments, the compute device 330 can be physically included in and/or on local machine(s) or device(s) or can be stored, run, executed, and/or otherwise implemented in and/or on remote machine(s) or device(s). For example, the compute device 330 (or a portion or component thereof) can be and/or can include, but is not limited to, PC(s), laptop(s), tablet(s), mobile device(s) (e.g., a smart phone, wearable, etc.), server(s), workstation(s), and/or the like. In some embodiments, the compute device 330 or at least a portion thereof can be implemented as a virtual machine and/or virtual private server executed on and/or run as an instance or guest on a physical machine and/or cloud platform like Microsoft Azure®, Amazon® web services, IBM® cloud computing, etc. In some embodiments, the compute device 330 may be associated with a healthcare service provider such as a doctor, hospital, or medical center, and/or a user such as an emergency responder, a healthcare professional, a patient, and/or the like.

The compute device 330 includes a processor 332, a memory 334, an input/output (I/O) device 336, and a communication device 338. The processor 332, the memory 334, the I/O device 336, and the communication device 338, are in communication (e.g., via a system bus or the like) allowing instructions, signals, data, etc. to be transmitted therebetween.

The processor 332 can be and/or can include one or more data processing units, signal processing, engines, modules, devices, circuits, controllers, etc. configured to execute the operations of the compute device 330. In some embodiments, the processor 332 can be a hardware based integrated circuit (IC), or any other suitable processing device configured to run and/or execute a set of instructions or code. For example, the processor 332 can be one or more data processors, image processors, an analog signal processor, a mixed-signal processor, a general-purpose processor, a central processing unit (CPU), an accelerated processing unit (APU), a graphics processing unit (GPU), a digital signal processor (DSP), an application specific integrated circuit (ASIC), a field programmable gate array (FPGA), a programmable logic array (PLA), a complex programmable logic device (CPLD), a programmable logic controller (PLC), a machine learning processors, a deep learning processor, a finite state machine (FSM), and/or the like. The underlying device technologies may be provided in a variety of component types such as metal-oxide semiconductor field-effect transistor (MOSFET) technologies like complementary metal-oxide semiconductor (CMOS), bipolar technologies like generative adversarial network (GAN), polymer technologies (e.g., silicon-conjugated polymer and metal-conjugated polymer-metal structures), mixed analog and digital technologies, and/or the like. As described in further detail herein, the processor 332 is configured to execute instructions, code, modules, applications, etc. stored in the memory 334.

The memory 334 stores instructions that are executed by the processor 332. The memory 334 can be any suitable volatile or non-volatile memory such as, for example, a random-access memory (RAM)—inclusive of any type/subtype of RAM, a read-only memory (ROM)—inclusive of any type/subtype of ROM, a memory buffer, a flash memory, and/or the like or combinations thereof. In some instances, the memory 334 can store, for example, one or more software programs and/or code that can include instructions to cause the processor 332 to perform one or more processes, functions, and/or the like associated with the system 300. In some embodiments, the memory 334 can include extendable storage units that can be added and used incrementally. In some instances, the memory 334 can be remotely operatively coupled with a compute device (not shown). For example, a remote database device can serve as a memory (or at least a portion of a memory) and be operatively coupled to the compute device (e.g., via a network or the like).

The I/O device 336 can be and/or can include any suitable device(s), interface(s), port(s), etc. that can allow the compute device 330 to receive an input and/or to provide an output. For example, in some implementations, an input can include a port or wireless communication device configured to communicate with and/or receive input from a keyboard, mouse, and/or any other peripheral device. In some implementations, an output can include a port or wireless communication device configured to communication with and/or provide output to an audio device, a display device, a haptic device, and/or any other suitable device. For example, an I/O device can be, can include, and/or can be configured to at least partially control a display that can provide at least a portion of a user interface for a software application (e.g., a mobile application, a PC application, an internet web browser, etc.) installed and/or executed on or by the compute device 330 (or the processor 332 thereof). In such implementations, the display can be, for example, a cathode ray tube (CRT) monitor, a liquid crystal display (LCD) monitor, a light emitting diode (LED) monitor, and/or the like.

The communication device 338 can be any suitable device(s) and/or interface(s) that can communicate with and/or via a network (e.g., the network(s) 305). The communication device can include one or more wired and/or wireless interfaces, such as, for example, Ethernet interfaces, optical carrier (OC) interfaces, and/or asynchronous transfer mode (ATM) interfaces. In some embodiments, the communication device can be, for example, a network interface card and/or the like that can include at least an Ethernet port and/or a wireless radio (e.g., a Wi-Fi® radio, a Bluetooth® radio, near field communications (NFC) radio, etc.). In some embodiments, the communication device can include and/or can be in communication with a device that includes one or more communication antenna (e.g., a satellite antenna, wireless radio antenna, and/or the like). In some embodiments, the communication device can be configured to receive/send signals and/or data from/to any number of devices, data sources, assets, machines, controllers, sensors, systems, etc. via the network(s) 305.

The optional external data source(s) 340 shown in FIG. 3 can be one source or multiple sources storing and/or otherwise configured to store information. The external data source(s) 340 can be and/or can include one or more repositories, databases, servers, and/or other data sources that is/are in communication with the compute device 330 and the sensing device(s) 310 via the network(s) 305. In some implementations, the external data source(s) 340 store and/or are configured to store information associated with the patient P, health-related data, machine learning and/or AI models and/or training data sets, and/or the like. For example, the external data source(s) 340 can include and/or can store data associated with training data sets configured to train any of the machine learning models described in further detail herein; information associated with the patient P (e.g., demographics information, age, sex, weight, height, health history, diagnoses, health trends, health predictions, treatment programs or modalities, sensitivities, allergies, etc.); non-patient-specific data such as environmental data (e.g., that may impact health), community or other broader-population health-related data; and/or any other suitable data. Moreover, the external data source(s) 340 can include and/or can store relationships, associations, correlations, etc. associated with such patient-specific data and/or non-patient-specific data. In some embodiments, the external data source(s) 340 can include specific information associated with the health of the patient P that can be used for correlation. For example, the external data source(s) 340 can include information associated with fatigue, pulmonary edema, peripheral edema, shortness of breath, and/or other characteristics that can be associated with the respiratory and/or cardiac systems of the patient P.

In some implementations, the optional external data source(s) 340 can be configured to send, via the network(s) 305, patient-specific data, non-patient-specific data, and/or any other suitable data to at least the compute device 330, which in turn, can process, analyze, and/or otherwise use the data to, for example, define, update, train, and/or execute one or more machine learning algorithms or models associated with defining and/or generating one or more diagnoses or diagnostic predictions (e.g., specific to the patient P or associated with a broader population). In addition, the external data source(s) 340 can be configured to receive, via the network(s) 305, patient-specific data, non-patient specific data, and/or any other suitable data from the compute device 330 and/or the sensing device(s) 310. In response, the external data source(s) 340 can store the data and/or otherwise update data already stored.

The sensing device(s) 310 shown in FIG. 3 can be and/or can include one or more device(s) configured to sense, detect, monitor, and/or measure bio-signals associated with one or more characteristics (e.g., physiological characteristics, pathophysiological characteristics, and/or the like, or combinations thereof) inside or outside of the body of the patient P. The sensing device(s) 310 can be implanted in the body of the patient P or can be disposed outside of the patient P. For example, the sensing device(s) 310 can be and/or can include one or more implantable diagnostic, sensing, and/or monitoring device(s) or can be and/or can include one or more implantable treatment device such as an ICD, a CRT-D, a pacemaker, an implanted electrical stimulator, and/or the like. Alternatively or in addition, the sensing device(s) 310 can include a wearable device, such as a smart watch, a fitness tracker, an insulin pump, a thermometer, a pulse oximeter, a smart ring, and/or the like. In some embodiments, the sensing device(s) 310 can include a combination of devices. For example, the sensing device(s) 310 can include one or more implantable device(s) and one or more wearable device(s).

In some embodiments, the sensing device(s) 310 may be implanted in an anterior mediastinum of a patient. Implanting in the anterior mediastinum can allow for the sensing device(s) 310 to sense, detect, monitor, and/or measure bio-signals associated with the anatomy of the patient P in the anterior mediastinum including the function of the heart and/or the pulmonary system. For example, disposing the sensing device(s) 310 in the anterior mediastinum can allow for sensing changes in pressure associated with the function of the heart due to the heart expanding and contracting while pumping blood and/or the function of the pulmonary system due to the lungs expanding and contracting during inspiration/expiration.

As described in detail herein, the sensing device(s) 310 are configured to monitor and/or measure multiple characteristics (e.g., including electrical characteristics, mechanical characteristics, and/or the like) associated with the patient P. Collecting data and/or information about the characteristics can allow the sensing device(s) 310 to monitor the health of the patient over time and/or determine or define any suitable diagnosis or diagnostic prediction. In some instances, the multiple characteristics can be correlated to allow for a more accurate and precise diagnostic prediction than a diagnostic prediction using just one characteristic. In some embodiments, the sensing device(s) 310 can be configured to monitor the patient P continuously, periodically, and/or sporadically. For example, the sensing device(s) 310 can be configured to sense patient characteristics during a predetermined number of cardiac cycles each day. In some embodiments, the sensing device(s) 310 are configured to automatically sense patient characteristics. In some embodiments, the sensing device(s) 310 are configured to sense patient P characteristics during a predetermined action associated with the patient (e.g., exercising, sleeping, cardiac and/or respiratory training, rehabilitation, recovery, etc.). In some embodiments, the sensing device(s) 310 are configured to sense patient P characteristics based on the circadian rhythm of the patient P.

The sensing device(s) 310 include a set of sensors 320 and optionally include a processor 312, a memory 314, an input/output device 316, and a sensor interface 318. The processor 312 can be configured to execute the operations of the sensing device(s) 310. The processor 312 can be, for example, a hardware based integrated circuit (IC), or any other suitable processing device configured to run and/or execute a set of instructions or code. For example, the processor 312 can be a general-purpose processor, a CPU, an APU, an ASIC, a FPGA, a PLA, a CPLD, a PLC, and/or the like. The processor 312 can be operatively coupled to the memory 314 through a system bus (for example, address bus, data bus, and/or control bus). As described in further detail herein, the processor 312 is configured to execute instructions, code, modules, applications, etc. stored in the memory 314.

The memory 314 stores instructions that are executed by the processor 312. The memory 314 can be any suitable volatile or non-volatile memory such as, for example, any type/subtype of RAM, any type/subtype of ROM, a memory buffer, a flash memory, and/or the like or combinations thereof. In some instances, the memory 314 can store, for example, one or more software programs and/or code that can include instructions to cause the processor 312 to perform one or more processes, functions, and/or the like. As described in further detail herein, the memory 314 can include instructions that, when executed, cause the processor 312 to process and/or analyze data received from one or more data sources (e.g., sensor data received from any number of sensors and/or other data stores/sources), monitor, determine, define, diagnose, and/or predict a state, status, condition, event, anomaly, and/or trend (e.g., a physiological and/or pathophysiological state, status, condition, event, anomaly, and/or trend) associated with the patient P.

The input/output device 316 (e.g., I/O device 316) can be and/or can include any suitable device(s), interface(s), port(s), etc. that can allow the sensing device 310 to receive an input and/or to provide an output. The I/O device 316 is configured to allow the sensing device(s) 310 to communicate with one or more devices such as, for example, an external device, controller, server, etc. (e.g., a compute device, controller, etc. of a medical professional, the patient P, and/or the like). The I/O device 316 can be configured to send information via one or more networks using any suitable communication mode (e.g., Bluetooth, Low Energy Bluetooth, Wi-Fi, near field communication (NFC), and/or the like). The I/O device 316 can be configured to send information with a wired or wireless communication device.

The sensor interface 318 can be any suitable device(s), circuits, interfaces, etc. configured to allow and/or facilitate communication between the components of the sensing device(s) 310 and the sensors 320. Additionally, in some embodiments, the sensor interface 318 provides the sensors 320 with power. In some embodiments, the sensor interface 318 can preprocess signals output by and/or received from the sensors 320. Alternatively, in some embodiments, the I/O device 316 can be and/or can include the sensor interface 318 and can be configured to allow communication between the sensors 320 and at least the processor 312 and/or the memory 314 of the sensing device(s) 310.

Although not shown, the sensing device(s) 310 can optionally include and/or can otherwise be powered by a power system. In some embodiments, the power system can include at least one battery (e.g., LiPo, Li-ion, etc.). The at least one battery can be charged when the battery is low on power. In some embodiments, the power system includes a primary cell battery and a rechargeable battery. In some embodiments, the power system can be configured to charge automatically (e.g., via patient P movement) or wirelessly (e.g., via inductive charging).

The set of sensors 320 of the sensing device(s) 310 are configured to sense, detect, monitor, and/or measure bio-signals associated with one or more characteristics (e.g., physiological characteristics, pathophysiological characteristics, and/or the like, or combinations thereof) associated with the patient P or with a disease. In some embodiments, the sensing device(s) 310 can include sensors 320 configured to be implanted in the body of the patient P or disposed on or outside of the patient P′s body. More particularly, the sensing device(s) 310 can include at least one sensor configured to be implanted in the anterior mediastinum of the patient P.

The sensors 320 of the sensing device(s) 310 are configured to sense, detect, monitor, and/or measure any number of characteristics associated with the patient P. In some embodiments, the sensors 320 include at least one sensor that is implanted into the patient P. In some embodiments, the sensors 320 include at least one sensor that is configured to sense, detect, monitor, and/or measure any number of characteristics on or outside of the body of the patient P. In some embodiments, the sensors 320 can include a sensing lead, a pressure sensor, a photoplethysmography sensor (PPG) sensor (or other optical sensor), an oxygen saturation (SpO2) sensor, an electrocardiogram (or cardio electrogram) sensor, an accelerometer, a temperature sensor, an acoustic sensor, and/or the like. In some embodiments, each sensor of the sensing device 310 can be the same type of sensor or can be a different type of sensor. For example, the sensors 320 can include multiple pressure sensors configured to measure pressure (e.g., anterior mediastinum pressure, hemodynamic pressure, etc.) at different locations in the patient P. In some embodiments, the sensors 320 can include one or more pressure sensor configured to be implanted in the anterior mediastinum and configured to measure the pressure in the anterior mediastinum. In some embodiments, multiple sensors 320 can be co-located in or on the body of the patient P. In some embodiments, the sensors 320 are located in different locations in or on the body of the patient P. In some embodiments, at least one sensor of the sensors 320 are integrated into the sensing device(s) 310. In some embodiments, at least one sensor of the sensors 320 is located external to and/or apart from the diagnostic device(s) 310 (e.g., remote sensor, sensing lead, etc.) and is communicably coupled (e.g., via a wired connection, via a wireless connection, etc.) to the sensing device(s) 310.

In some embodiments, the sensors 320 include at least a first sensor configured to monitor and/or measure a first characteristic (e.g., a first bio-signal associated with and/or indicative of the first characteristic) and a second sensor configured to monitor and/or measure a second characteristic (e.g., a second bio-signal associated with and/or indicative of the second characteristic), where the first characteristic is different than the second characteristic. In some embodiments, the first characteristic and the second characteristic can be related, associated, and/or the like. For example, the first characteristic can be cardiac electrical signals and the second characteristic can be pressure signals in the anterior mediastinum). In some instances, the first and second characteristics can be related to and/or indicative of (alone or when considered together) a disease and/or other diagnoseable condition. It should be understood that the sensing device(s) 310 can include any number of additional sensors configured to sense and/or detect any suitable characteristic(s) (and/or bio-signal(s) associated with such characteristic(s)) associated with the patient P. In some embodiments, the sensors 320 can belong to a single sensing device(s) 310 or to multiple sensing device(s) 310. In some embodiments, the sensing device(s) 310 can generate sensor data that includes multiple signal vectors.

In some embodiments, the sensors 320 can be configured to sense, detect, monitor, and/or measure a different characteristic associated with an organ, or more generally, the patient P. In some implementations, one or more sensors 320 can be configured to sense or detect the same characteristic, thereby allowing for confirmation/verification of signal data and/or a desired degree of sensitivity and/or specificity in interpreting the signal data. In some embodiments, the sensors 320 can be configured to sense, detect, monitor, and/or measure characteristics (and/or bio-signal(s) associated with such characteristics) continuously, periodically, sporadically, or when a command is received (e.g., from the sensing device(s) 310).

In some embodiments, the sensors 320 can be configured to send and/or receive data from different components of the sensing device(s) 310. For example, the sensors 320 can be configured to send data to the processor 312, the memory 314, the I/O device 316, and/or the sensor interface 318. In the embodiments, communication between the sensors 320 and the other components of the sensing device(s) 310 may be facilitated by the sensor interface 318. Alternatively, in some embodiments, the sensors 320 can be in communication with the compute device 330. Additionally, in some embodiments, the sensor interface 318 provides the sensors 320 with power. In some embodiments, the sensor interface 318 can preprocess signals output by and/or received from the sensors 320. In some embodiments, the outputs of the sensor interface 318 and/or the sensors 320 can be calibrated based on the patient P, the position in the body of the patient P, and/or any other suitable calibration metric or combination of metrics, and/or the like. For example, the sensors 320 can be calibrated based on one or more known measurement(s) of patient characteristic(s). Calibration can include calibrating the output of the sensors 320 to align with known measurements of patient characteristics. For example, a cardiac electrical signal sensor used to measure, for example, cardiac electrical signals (e.g., electrocardiogram or cardiac electrogram) can be calibrated based on comparing and adjusting the sensor output to an external cardiac rate measuring device.

In some implementations, the sensing device(s) 310 can be used for short or long term monitoring of the characteristics associated with the patient P. Short term monitoring can include monitoring the health of the patient P to determine is a health event or anomaly is present (e.g., at a specific time). For example, short term monitoring can include determining if a triggering event is occurring to aid in treatment decisions. Long term monitoring can include monitoring over a period of time (e.g., days, weeks, months, years) to determine if there is an anomalous pattern, change, or trend in the health characteristics. In both the short and the long term monitoring, the sensing device(s) 310 are used to determine if a health event is occurring, to determine a diagnosis, to identify an anomaly, and/or the like.

In some embodiments, the sensing device(s) 310 can act as and/or form at least a portion of a remote intensive care unit (ICU). For example, the sensing device(s) 310 can be configured to sense, detect, monitor, and/or measure all or at least some of the characteristics that can be sensed, detected, monitored, and/or measured in an ICU including, but not limited to, cardiac cycle, pulmonary/respiratory cycle, nervous system, body temperature, glucose level, oxygen saturation, cardiac signals, sleep performance, recovery status, hemodynamic status, activity level, blood pressure, mediastinal pressure, sleep apnea, heart failure, heart valve failure, heart valve regurgitation (e.g., mitral regurgitation, tricuspid regurgitation, aortic regurgitation, and/or pulmonary regurgitation), stroke volume, respiratory rate, respiratory tidal volume, inspiration duration, expiration duration, and/or the like (or combinations and/or derivatives thereof). In some embodiments, the data provided by the sensing device(s) 310 (e.g., the remote ICU) can be correlated and/or used in conjunction with information and/or data stored or collected in an ICU environment in a hospital (i.e., not remote). Including the sensing device(s) 310 in a remote ICU environment allows for more precise diagnostics to be made in a remote setting than when only one characteristic is measured.

The sensing device(s) 310 can analyze, process, aggregate, correlate, etc. the data from the sensors to determine and/or define a status (e.g., a physiological status, a pathophysiological status, and/or the like, or combinations thereof) associated with the patient P. Furthermore, based on the determined and/or defined status, the sensing device(s) 310 can detect and/or determine the occurrence of health event, such as heart disease, a pulmonary disease, seizure, a heart attack, an overdose, tachycardia, arrhythmia, static and/or dynamic hyperinflation, dyspnea, change in heart failure status, and/or the like. In some embodiments, based on the determined and/or define status, the sensing device(s) 310 can determine a diagnostic status, such as a predicted diagnosis, an indication of a disease or disease state, progression of a disease state, a condition suitable for a given treatment/therapy (e.g., using an ICD, pacemaker, ventilator, oxygen supply, stimulator, etc.), and/or the like. In some embodiments, using the data from the sensors 320, which includes data from more than one sensor and/or otherwise associated with multiple characteristics, the status can be determined with more specification and/or accuracy than when determining a status using data from a single data source (e.g., from only one sensor detecting one bio-signal and/or characteristic).

In some embodiments, the process of the sensing device(s) 310 determining the status of the patient P can include, for example, comparing, correlating, verifying, confirming, and/or synchronizing the signal data received from the sensors 320. For example, the sensors 320 can determined and/or sense a first characteristic and a second characteristic. In some embodiments, the first characteristics and the second characteristic are different. In some embodiments, the sensing device(s) 310 can determine and/or sense additional characteristics and/or additional/other bio-signals. In some embodiments, the sensing device(s) 310 can also receive additional information associated with the patient which can also be used by the sensing device(s) 310 in determining the status.

The sensing device(s) 310 (and/or the processor 312 thereof) can determine the status based at least in part on comparing, correlating, verifying, confirming, and/or synchronizing the data from the sensors 320. To determine the status, the sensing device(s) 310 can, for example, determine if data associated with at least one characteristic of the characteristics measured or sensed by the sensors 320 indicates a status. Based on the correlated and/or associated characteristics, the sensing device(s) 310 can determine if other determined or sensed characteristics are as expected based on the status. If the characteristics are not as expected, the sensing device(s) 310 can modify, update, and/or augment the status. In some embodiments, one or more machine learning algorithms may be employed to define, determine, confirm, verify, correlate, predict, etc. expected status(es) and/or output(s) for a given status. In some embodiments, one or more machine learning algorithms may be employed to define, determine, confirm, verify, correlate, predict, etc. a status based at least in part on one or more relationships and/or a degree of correlation between, for example, data received from the sensors 320 (and/or any other suitable data source).

The sensing device(s) 310 (and/or the processor 312 thereof) can be configured to process data received from one or more data sources (e.g., sensors, etc.) and monitor the associated characteristics for determining, predicting, identifying an anomaly, and/or diagnosing a state, status, and/or event (e.g., a physiological and/or pathophysiological state, status, and/or event) of the patient P. For example, the memory 314 can include instructions that can cause the processor 312 to analyze sensor data received from any number of sensors, devices, data stores, etc. and to determine and/or define one or more predictions, diagnosis, confirmation of arrhythmia, etc. These instructions can be updated and/or refined based on learnings from previously received data. For example, data (e.g., sensor data and/or any other suitable data) can be provided as an input into one or more machine learning models to update, refine, and/or train the model(s) to provide an output associated with monitoring the health of the patient P and/or to determine a predicted health condition, state, status, and/or event based on the data. In some embodiments, the compute device 330 is configured to receive the data from the sensing device(s) 310 and update, refine, and/or train the machine learning model(s). Once updated, the compute device 330 can send, via the network(s) 305, the updated/trained model(s) to the sensing device(s) 310, which in turn, can update, supplement, replace, overwrite, etc. instructions, models, algorithms, and/or processes stored in the memory. In some instances, the memory 314 can include the updated machine learning model, which the processor 312 can execute to processes the data received from the sensors 320 or other data sources to provide a prediction and/or diagnosis. In some instances, the memory 314 can include one or more look-up tables (defined at least in part by one or more machine learning model(s)), which the processor 312 can access to select the most likely predicted state, status, event, etc. (e.g., a predicted diagnosis) based on the data.

For example, the sensing device(s) 310 (and/or the processor 312 thereof) can be configured to determine one or more cardiac parameters and/or one or more respiratory parameters based on the signals from the sensors 320. For example, based on the sensors 320 measuring a hemodynamic output, cardiac parameters can be determined such as left ventricular end-systolic pressure, left ventricular pressure rate, stroke volume, right atrial pressure, pulmonary capillary wedge pressure, valve function, and/or the like. For example, stroke volume can be determined by the change in pressure measured in the anterior mediastinum being correlated with the pressure changes associated with the beating of the heart. As another example, based on the sensor 320 measuring a respiratory output, pulmonary characteristics can be determined such as end-expiratory pressure, end-inspiratory pressure, tidal volume, expiratory time, inspiratory time, respiratory rate, and/or the like.

In some embodiments, the parameters can be used to determine and/or correlate with symptoms of a condition. For example, certain cardiac parameters can be associated with symptoms of heart failure. Specifically, high or increased right atrial pressure can be associated with edema or worsening edema, high or increased pulmonary capillary wedge pressure can be associated with a shortness of breath or worsening shortness of breath, and low or reduced stroke volume (and/or cardiac output) can be associated with fatigue or worsening fatigue. Additionally, the cardiac parameters can be associated with the function of the cardiac valves (e.g., aortic or mitral valve regurgitation can be associated with new or worsening shortness of breath, fatigue, swelling, arrhythmia, and/or the like). The parameters (or changes in the parameters), when monitored over a period of time, can indicate, for example, heart failure or worsening heart failure (or heart valve failure). In some instances, based at least in part on the parameters or changes in the parameters, the sensing device(s) 310 (and/or the processor 312 thereof) can detect, determine, and/or predict onset or worsening conditions, states, events, etc. (e.g., even before the relevant symptoms are experienced by the patent). In other words, before the relevant symptoms appear, the sensing device(s) 310 (and/or the processor 312 thereof) can predict the worsening of relevant symptoms, conditions, states, etc., based on the changes in the parameters, which in turn, can allow a physician to take measures to address these parameters with the patient before symptoms appear and/or otherwise become more debilitating. In some embodiments, the pressure in the anterior mediastinum can be monitored over a period of time to determine whether and/or how the pressure therein is changing. In some embodiments, the change can be associated with a fluid load, a mediastinal neoplasm, and/or the like.

The sensing device(s) 310 (and/or the processor 312 thereof) can be configured to perform one or more steps associated with preprocessing the signals from the sensors 320. For example, the memory 314 can include and/or store instructions that when executed by the processor 312 perform one or more preprocessing steps that can include amplification, filtering, analog to digital conversion, signal recognition and detection, synchronization, and/or the like. Preprocessing can include generating one or more curve based on the signals form the sensors 320. For example, the curves can include a cardiac hemodynamic curve, a respiratory function curve, an anterior mediastinum pressure curve, and/or the like. In some embodiments, the curves can be associated with pulmonary ventilation function, tidal volume, cardiac stroke volume, pulmonary capillary wedge pressure (PCWP), right arterial pressure, central venous pressure, expiration time, inspiration time, rate and resistance during inspiration/exhibition, and/or the like. In some embodiments, generating the curve can be based on one or more signal from the sensors 320. For example, the cardiac hemodynamic curve can be based on both a cardiac electrical signal and a pressure signal, synchronized to determine key events, while a respiratory function curve and an anterior mediastinum pressure curve can be based on the pressure signal.

The sensing device(s) 310 (and/or the processor 312 thereof) can be configured to perform one or more steps associated with monitoring based on the signals and determining a status including one of a diagnosis, anomaly, and/or health event, with or without preprocessing. The monitoring can be associated with a bodily process of the patient P, a system of the body of the patient P, a portion of the body of the patient P, sensor data, and/or the like. Determining the status can include comparing an output from the sensor 320 and another output from the sensors 320 to recognize and confirm the status. For example, if the output from the sensors 320 appears to indicate an irregular heartbeat, but another output from the sensors 320 does not appear to indicate an irregular heartbeat, then the output indicating the irregular heartbeat may be determined to be a false positive. Determining and/or identifying false positives can allow the sensing device(s) 310 to decrease the number of undesired notifications and/or alerts for the patient P and is more specific than if using an output from just one of the sensors 320. If multiple outputs from the sensors 320 indicate the same and/or complementary status, that status is determined, corroborated, and/or confirmed.

For example, in some embodiments, the memory 314 can include instructions associated with correlating a first output from the sensors 320 and a second output from the sensors 320. In some embodiments, the first output and the second output can be complementary and confirmatory functions, characteristics, states, etc. For example, the first output can be an electrical characteristic (e.g., cardiac electrical data) and the second output can be a mechanical characteristic (e.g., cardiac mechanical data and/or any other mechanical data sensed or detected within a portion of the body of the patient P such as the anterior mediastinum), which can allow the processor 312 to correlate and/or verify an electrical characteristic with or to an expected mechanical characteristic. As another example, the first output can be respiratory/pulmonary data (e.g., respiratory rate, period, cycle, lung volume, inspiration/expiration characteristics, etc. as determined and/or sensed by a pressure sensor and/or the like), and the second output can be oxygen saturation data (e.g., from an internal or external pulse oximeter and/or the like). In some embodiments, the memory 314 can include instructions associated with correlating the outputs from the sensors 320 with imaging data generated and/or received from the external data source(s) 340. For example, the imaging data can include imaging data associated with the changes in volume of the anterior mediastinum during function of the heart and/or the lungs.

In some embodiments, the data from the sensors 320 can be used with and/or correlated with imaging data from one or more imaging devices. For example, the imaging data can include, for example, imaging data generated using coronary computed tomography angiography (CCTA), ultrasound imaging, computed tomography (CT), x-ray imaging, magnetic resonance imaging (MRI), positron emission tomography (PET) scan, and/or the like. In some embodiments, the imaging device can be used and the imaging data from the imaging device can be stored in a memory, database, and/or the like. The imaging data, in turn, can be sent to the system 300 (e.g., the sensing device(s) 310 thereof).

As described above, the process of determining a status of the patient P can utilize and/or otherwise employ one or more artificial intelligence algorithm(s) (e.g., a machine learning model(s) and/or algorithm(s)) to determine and/or predict the status. For example, the machine learning model(s) can be trained to determine the status based at least in part on outputs from the sensors 320. In some embodiments, the compute device 330 can be used to define the machine learning models and/or to perform the training, refining, etc. In some embodiments, the machine learning model(s) and/or algorithm(s) (referred to herein as machine learning “model” or “algorithm” interchangeably) can be configured to filter out or recognize physiological activity (e.g., breathes, heartbeats, physical activity, etc.) that were determined or missed during an initial determination. In some embodiments, the machine learning algorithm(s) can correlate, compare, verify, confirm, and/or otherwise process the outputs from sensors 320 to determine a pattern corresponding to a status, etc. In some embodiments, the machine learning algorithm(s) can learn from the outputs of the sensors 320 to determine long-term correlation(s), patterns, trends, and/or changes thereof between the outputs of the sensor 320. In some embodiments, the long-term correlation and changes can be used to determine a long-term status such as identifying anomalies and/or determining a diagnosis that may not be apparent in a short-term period.

In some embodiments, the process of determining the status can utilize and/or otherwise employ one machine learning model (e.g., a multivariate model). In some embodiments, the process of determining the status can utilize and/or otherwise employ more than one machine learning model. In some embodiments, different outputs from the sensors 320 can be processed by different machine learning models. For example, a first output from a first sensor (e.g., including data associated with cardiac electrical signals radiating from the heart) can be processed by a first machine learning model and a second output from a second sensor (e.g., including data associated with, for example, pressure changes in an anterior mediastinum of the patient P) can be processed by a second machine learning model. In some embodiments, a machine learning model can be associated with multiple types of sensor outputs. In some embodiments, information from the external data source(s) 340 can also be processed (independently or in conjunction with any other model) by a machine learning model. In some embodiments, each machine learning model can be configured to output, based on the data it receives/processes, a prediction associated with a status of the patient. In some embodiments, the compute device 330 can be used to define, train, and/or refine the machine learning models and/or to define, train, and/or refine one or more associations, correlations, aggregations, etc. of the outputs of multiple machine learning models. As such, steps that involve to use of a high degree of processing power can be performed and/or executed by the compute device 330, while the sensing device(s) 310 can perform steps that implement and/or use the previously defined models, which generally do not require significant processing power (and therefore, are suitable for execution by a device implanted in the body of the patient P.

In some embodiments, a machine learning model can be used to correlate, confirm, and/or verify the outputs from the sensors 320. In some embodiments, the machine learning model used to correlate the outputs from the sensors 320 may be different than the machine learning model(s) used to initially analyze and/or process the output from the sensors 320. In some embodiments, a machine learning model can be used to aggregate the correlated data. In some embodiments, the machine learning model used to aggregate the outputs from the sensors 320 may be different than the machine learning model(s) used to initially analyze and/or process the output from the sensors 320 and to correlate the data. For example, determining the status can include a series of machine learning models that are used to process data from the sensors 320, correlate the processed data, aggregate the sensor data, and then determining the status based on the data.

In some embodiments, the machine learning models used in determining the status can be the same type of machine learning model. In some embodiments, at least two of the machine learning models used in determining the status can be different machine learning models. For example, the machine learning models can include a support vector machine, deep learning, convolution neural network, recurrent neural network, regression, and/or the like. The type of machine learning model used can be associated with the type of data being processed, the source, and/or the like. For example, it may be desirable to process raw data from the sensors 320 with a first type of machine learning model and to correlate the outputs from the sensors 320 with a second type of machine learning model. As another example, it may be desirable to process the data from the sensors 320 with a first type or first group of machine learning model(s) and to correlate the outputs from the first type or group of machine learning model(s) using a second type or second group of machine learning model(s).

In some embodiments, the machine learning algorithm(s) described herein can use weights, scores, or similar metrics to determine the diagnostic status and/or to make a diagnostic prediction. For example, certain types of data, data from certain sources, data from different sensor locations, data from different sensor types, and/or the like can have a higher or otherwise different score than other data. In some embodiments, the weights, scores, or similar metrics may be associated with the relevance of the data or the predictive value of the data (or at least expected predictive value). For example, data associated with characteristics related (e.g., directly) to the status being determined can have a higher score, where a higher score indicates a higher relevance, than characteristics that are less related (e.g., indirectly). For example, if the status is a cardiac status, direct or inferred cardiac electrical and/or mechanical measurements can have a higher score than, for example, a temperature measurement. Alternatively, if the status is not directly related to the heart, cardiac electrical and/or mechanical measurements may be less determinate and therefore, given a lower score or weight.

In some embodiments, the machine learning models can be configured to cluster relevant signals from the sensors 320 (and/or any other data source). For example, the machine learning models can be configured to cluster signals that are relevant to a status or multiple statuses (e.g., physiological status(es), pathophysiological status(es), and/or the like, or combinations thereof) that the sensing device(s) 310 are configured to determine. Clustering signals can allow for the sensing device(s) 310 to only process and/or correlate data and/or signals from the sensors 320 and/or the external data source(s) 340 that is relevant to the desired status(es). For example, if a user indicates that only certain status determinations are desired, the sensing device(s) 310 may only process the data that has been clustered as being relevant to the status indicated as desired.

In some embodiments, the machine learning models can be configured to use image data from an imaging device (e.g., coronary computed tomography angiography (CCTA) imaging data, and/or any other suitable image data) to improve and/or correlate predictions and/or diagnoses based on data from the sensors 320. Alternatively, data output by the sensors 320 (and/or predictions and/or diagnoses based on the data) can be used to improve and/or correlate determinations, inferences, predictions, diagnoses, etc. based on the image data. The image data can be used to determine if a condition is occurring that other sensors may not indicate. For example, the image data can show if an artery is blocked, if a tumor is present (or show characteristics associated with a tumor), if a bone is damaged or broken, and/or the like. In some embodiments, machine learning algorithm(s) can be used to correlate and/or augment the data from the sensors 320 with the image data. In some embodiments, the machine learning algorithm(s) can be used to make predictions on what the image data is showing and how it relates to a status.

In some embodiments, the memory 314 can store instructions for determining whether a health event (e.g., an adverse health event that may be undesirable or dangerous to the patient P) is occurring based on the status or predicted status of the patient P. In some embodiments, the processor 312 may execute a machine learning algorithm, which may be the same or different than that used in refining, determining, and/or predicting the status, for determining if a health event is occurring. The memory 314 can further include and/or store instructions for execution by the processor 312 associated with determining the type of health event. For example, the processor 312 determining the type of health event can include correlating the status with known statuses associated with health events. In some embodiments, the memory 314 can further include and/or store instructions for determining if the health event is an adverse health event. Similarly stated, the memory 314 can include and/or store instructions for determining and/or distinguishing an adverse health event from, for example, a normal or non-adverse health event that may present and/or may be associated with one or more similar characteristics. In some embodiments, the processor 312 can determine the type of adverse health event (e.g., any of the adverse health events described herein). In some embodiments, the processor 312 may execute one or more machine learning algorithms, which may be the same or different than the other machine learning algorithms described herein, for determining the type of health event.

In some embodiments, the memory 314 can store instructions that cause the processor 312 to determine and/or define a diagnosis and/or diagnostic prediction (e.g., a result of a diagnostic test of the patient P) based on the status, health event, and/or additional information associated with the patient P. In some embodiments, the processor 312 may execute a machine learning model, which may be the same or different than that used in refining, determining, and/or predicting the status, for determining if a health event is occurring. In some embodiments, the sensing device(s) 310 can be used for both determining if a health event is occurring and for determining a diagnostic status or one of determining if a health event is occurring or for determining a diagnostic status.

In some embodiments, the memory 314 can store instructions for determining a cardiac and/or pulmonary status based on the outputs of the sensors 320 and/or the cardiac/pulmonary curves. In some embodiments, the processor 312 may execute a machine learning model for assessing the cardiac and/or pulmonary status to determine if an anomaly is present. In some embodiments, the machine learning model can be used during monitoring one or more of a cardiac and/or pulmonary parameter to determine the cardiac and/or pulmonary status.

While the memory 314 is described above as storing one or more machine learning models (executed by the processor 312), which can be used to determine the occurrence of a health event, anomaly, and/or to make a diagnosis or a diagnostic prediction for a patient, in other implementations, the memory 314 can store any number of processes, functions, instructions, etc. (executed by the processor 312) that is/are not machine learning model(s). In some implementations, for example, any of the machine learning models described herein can be used to determine and/or define an algorithm and/or a set of relationships, correlations, etc. for processing data associated the characteristic(s) (e.g., physiological characteristic(s), pathophysiological characteristic(s), and/or the like, or combinations thereof). In this manner, the processor 312 can execute the defined and/or determined algorithm without having to execute the machine learning model(s), which in turn, may reduce processing load and/or time. In other implementations, a look-up table may be defined (e.g., using any of the machine learning models described herein and/or via any other suitable process). In such implementations, the processor 312 can reference the look-up table to determine, based on the data associated with the characteristic(s), a diagnostic status for the patient and/or whether a heath event is occurring (or a likelihood thereof).

In some embodiments, the memory 314 can store instructions that cause the processor 312 to generate a notification associated with the status, health event, and/or the diagnostic status. The notification can be used to indicate to a user of a status for monitoring purposes, or how a status relates to a predetermined threshold. The notification can be used to indicate to a user that a health event is occurring. The notification can be used to indicate to a user that the diagnostic status is ready for review. In some embodiments, the notification can be configured for sending to an emergency responder, a medical provider, secondary user (e.g., other than the patient P), and/or the like. The notification can be used so that corrective action can be taken if necessary. Generating the notification can be used when the sensing device(s) 310 are used for monitoring purposes to alert a recipient of a detected change or anomaly.

In some embodiments, the memory 314 can store instructions that cause the processor 312 (and/or the I/O device 316) to indicate, and/or generate data indicating, that a health event is occurring. For example, the sensing device(s) 310 (or processor 312 and/or the I/O device 316 thereof) can define data or one or more signals associated with a visual, auditory, and/or physical notification that indicates that the health event is occurring. For example, sensing device(s) 310 can cause and/or instruct a display to display that the health event is occurring, an alarm to sound or other be graphically represented on the display, a vibration to occur, and/or the like. Similarly, the sensing device 310 can cause and/or can instruct the display to represent results from and/or data associated with a diagnostic test. For example, the display can display a value associated with a diagnosis and/or an indication that a diagnostic test is complete, a light can indicate that a diagnostic status, a vibration can indicate a diagnostic status, and/or the like. In some implementations, one or more signals, notifications, and/or the like can be sent (e.g., via the network(s) 305) to the compute device 330. For example, the sensing device 310 can be configured to send a signal indicative of an alarm notification to the compute device 330. The alarm notification can indicate a type of health event, such as a heart attack, seizure, overdose, etc., so that a user associated with the compute device 330 can respond to the health event.

In some embodiments, the sensing device 310 can receive input signals and/or commands from a device external to the body of the patient P and can, in turn, provide the input signals to the processor 312 of the sensing device(s) 310. For example, the sensing device(s) 310 (and/or the I/O device 316 thereof) can receive input signals from one or more devices (e.g., touchscreen, keyboard, mouse, buttons, etc.) representing inputs by a user (e.g., doctor, patient, caretaker, etc.). The inputs, in turn, can represent information associated with a diagnostic status, the patient P, a health event, and/or the like. In some embodiments, if the user receives a notification that a health event is occurring (e.g., from or generated by the I/O device 316), the user can indicate that an alarm system should be sent to an emergency responder, medical professional, and/or the like. In some embodiments, the sensing device 310 can be configured to receive data associated with the patient P such as from the external data source(s) 340 and/or the compute device 330, which may alter and/or may otherwise be used in the operation of the sensing device(s) 310 and/or in the determination of a diagnosis or in a treatment decision, etc.

In some implementations, the sensing device 310 can send information to a user and/or a compute device controlled by a user regarding the sensing device(s) 310 and/or an operational status of the sensing device(s) 310 such as battery level, notifications, device status, health status, change(s) in health status, and/or the like. The sensing device 310 can also send information regarding the patient P, such as real-time or substantially real-time information, data, and/or other signals associated with the patient P (e.g., data from the sensors 320, and/or any other suitable data). In some embodiments, the sensing device 310 can receive signals for augmenting and/or at least partially controlling the operation of the sensing device(s) 310. For example, the sensing device 310 can receive signals (e.g., from the compute device 330, the external data source(s) 340, and/or any other device) associated with patient information and/or other operational instructions for determining health events, diagnoses, and/or the like. In some embodiments, the sensing device 310 can be configured to send information and/or signals to the compute device 330 and/or server that is/are configured to perform machine learning processes. The sensing device 310 can also receive information from the compute device 330 and/or the server that may include instructions, firmware, updates, etc. based on an output from a machine learning model. In some implementations, the sensing device 310 can be configured to send a notification and/or other suitable data indicative of a change in a health state, health condition, disease state, pathology, etc. For example, the notification and/or data can include an indication of an emerging or worsening characteristic, state, condition, disease, etc. and a recommendation, instruction, and/or prescription to change a medical treatment such as increase a dosage of a prescribed medication, update for a pacemaker operating state or profile, and/or any other suitable medical treatment.

In some embodiments, the compute device 330 described above may be configured to execute and/or perform any of the processes and/or any suitable portion of the processes described in reference to the sensing device(s) 310 and/or otherwise associated with the system 300. For example, certain operations described as being performed by the sensing device(s) 310 can be executed by the compute device 330. In some implementations, the sensing device(s) 310 may be configured to preprocess data from the sensors 320 while the compute device 330 is configured to process, correlate, and/or otherwise use the preprocessed data. In some embodiments, the compute device 330 can be used to complete certain processes that may be too resource intensive (e.g., battery power, memory, processing power, etc.) for the sensing device(s) 310 and/or to otherwise decrease the computational resources needed by the sensing device(s) 310. In some embodiments, for example, the compute device 330 can be used to define, train, execute, and/or update, via the processor 332, one or more of the machine learning models, processes, and/or algorithms described above, which in turn, can be used by the sensing device(s) 310 using a given set of data associated with a patient.

In such embodiments, the compute device 330 can receive data from a large number of patients and/or large amounts of data from, for example, the external data source 340, which in turn can be used to train the machine learning models, processes, and/or algorithms. Once defined, trained, updated, etc., the machine learning models, processes, and/or algorithms, can be executed by the sensing device(s) 310 using a given set of data associated with a given patient P. In this example, the amount computational resources associated executing the machine learning model for a given set of data associated with the patient P is significantly less than the computational resources associated with defining, training, updating, and/or refining the machine learning model. In some embodiments, the compute device 330 may be configured to send commands and/or outputs to the sensing device(s) 310 so that the sensing device(s) 310 can, in turn, alter the functionality of the sensing device(s) 310, display information on the I/O device 316, and/or the like. In some embodiments, the compute device 330 may be configured to send commands and/or outputs to the sensing device(s) 310 that can cause the sensing device(s) 310 to update firmware, data stored in one or more repositories (e.g., databases), and/or instructions stored in the memory 334. As such, the sensing device(s) 310 can adapt operations and/or determinations based on patient P changes over time.

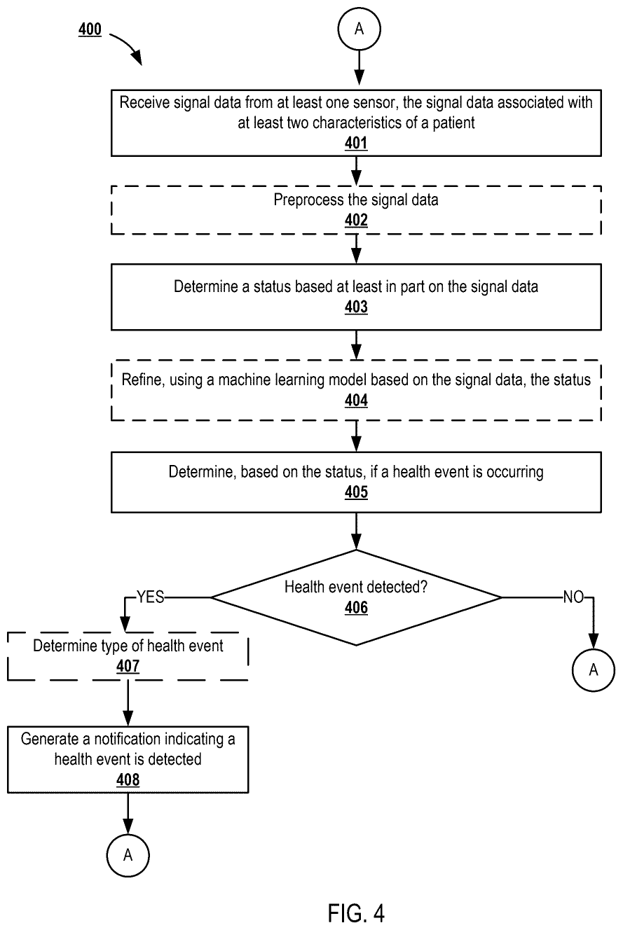

FIG. 4 is a flow chart depicting a method 400 for determining if a patient is having a health event, according to an embodiment. In some embodiments, the method 400 can be executed and/or performed by a diagnostic/monitoring system that is functionally and/or structurally similar to the diagnostic/monitoring system 300 of FIG. 3. In some embodiments, the method 400 can be executed by one or more devices. For example, the method 400 can be executed by the sensing device(s) 310 and/or the compute device 330. In some embodiments, the method 400 is configured to use at least two signals form at least two data source(s) (e.g., sensors) to determine if a patient is having a health event (e.g., sudden health event, chronic health event, etc.). For example, the at last two signals can include a cardiac electrical signal and a pressure signal associated with the anterior mediastinum. Using at least two signals reduces the likelihood of that a determining that a health event is occurring is a false positive. The method 400 includes generating a notification indicating that the health event is detected, which allows for a patient or a user to review the health event and take corrective action. The method 400 can be used by a patient that may be at-risk of a health event and is monitoring for a health event, thus allowing the patient and/or a caretaker or healthcare provider to act on the health event if the health event is determined to be occurring. The method 400 can be used to continuously (or semi-continuously) and/or periodically monitor the patient. For example, the method 400 can be used to monitor the patient during a high risk activity (e.g., high risk of a health event occurring) or continuously if the patient is at-risk for a health event.

The method 400 includes, at 401, receiving signal data from at least one sensor (e.g., functionally and/or structurally similar to any of the sensors described herein). The signal data is associated with at least two characteristics of a patient. In some embodiments, the at least one sensor can include any number of sensors. In some embodiments, the at least two characteristics can include a first characteristic and a second characteristic. The first characteristic can be associated with a status (e.g., physiological status(es), pathophysiological status(es), and/or the like, or combinations thereof) and the second characteristic can be associated with the same or different status. For example, the at least two characteristics can include an electrical characteristic associated with a status and a mechanical characteristic associated with the status. As another examples, the at least two characteristics can include a characteristic (electrical, mechanical, etc.) associated with a first status and a characteristic (electrical, mechanical, etc.) associated with a second status different from the first status. In some embodiments, both the signal data is measured in vivo and/or ex vivo. For example, the signal data can be measured in the anterior mediastinum, at the heart wall, or externally on the body of the patient. In some embodiments, step 401 may include receiving additional signal data from any number of additional sensors and/or any additional or other data sources (e.g., one or more ex vivo data sources). In some embodiments, step 401 may include receiving the signal data from an external data source (e.g., patient database, etc.). In some embodiments, step 401 may additionally include receiving additional information associated with the patient such as patient age, height, weight, demographic information, medical history, and/or the like.

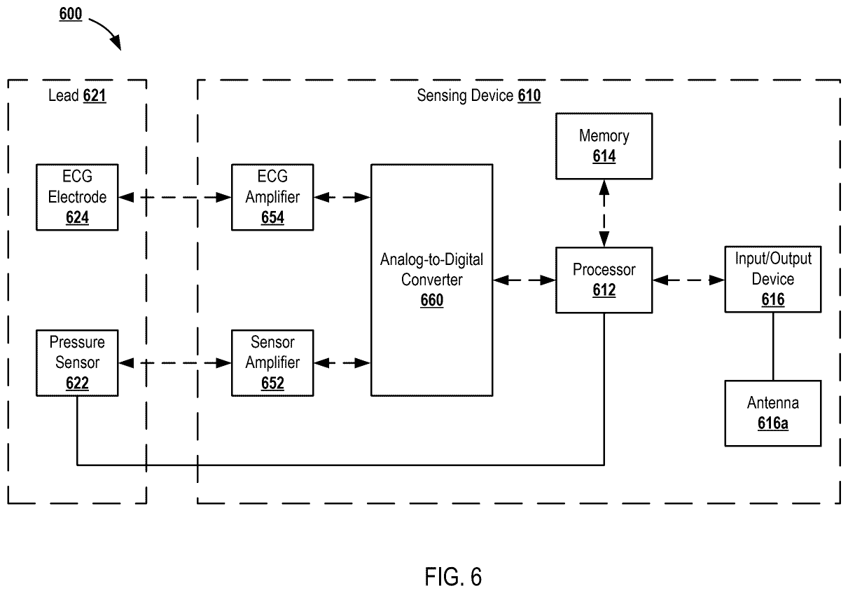

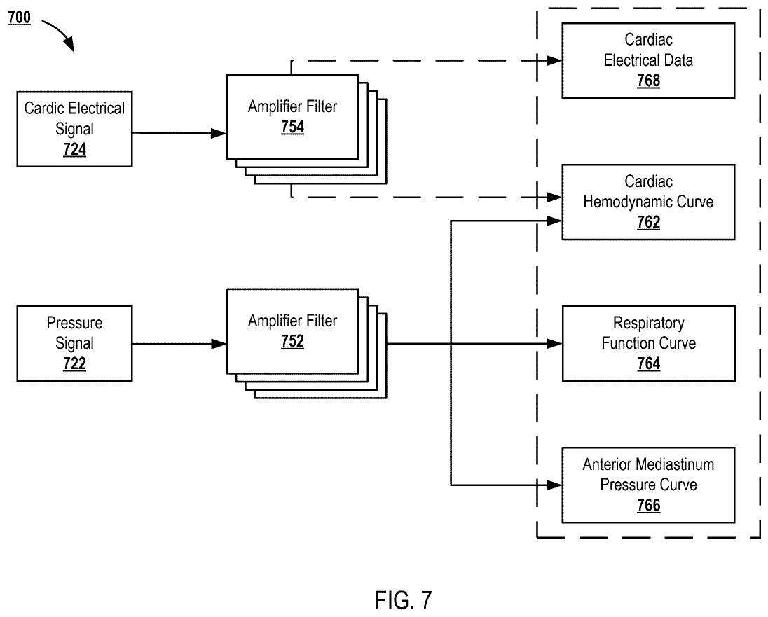

The method 400 optionally includes, at 402, preprocessing the signal data. Preprocessing can include noise reduction, filtering, amplification, normalization, conversion (e.g., analog-to-digital, etc.), synchronization, and/or the like. Preprocessing can prepare for the signals to be received by another component of the diagnostic/treatment system. For example, a sensor interface (e.g., functionally and/or structurally similar to any of the sensor interfaces described herein) can preprocess the signal data so that the signal data can be received by the compute device (e.g., structurally and/or functionally similar to any of the compute devices described herein). In some embodiments, the compute device may preprocess the first signal data and/or the second signal data to prepare the data for processing. In some embodiments, preprocessing can include generate one or more curve associated with the signal data. For example, the one or more curves can include a curve associated with cardiac function, respiratory function, anterior mediastinum pressure, and/or the like. Generating the curves is described in further detail in reference to FIGS. 6-7.

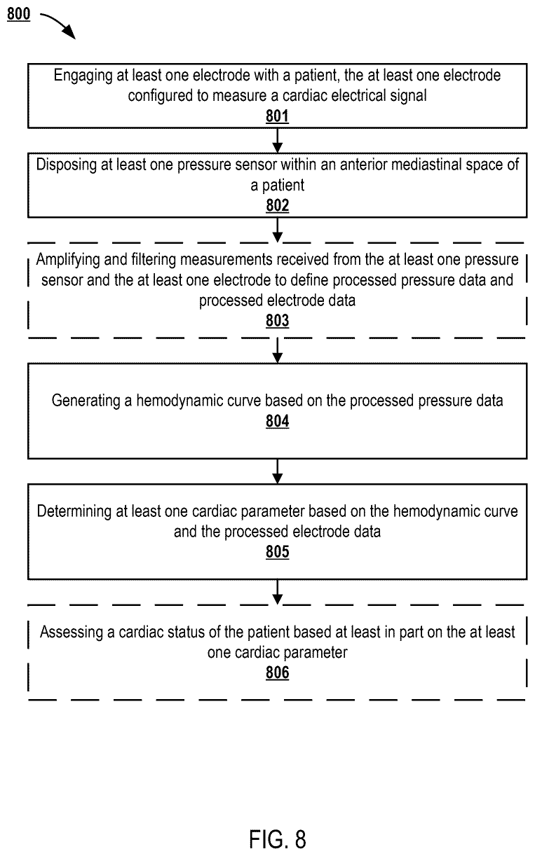

The method 400 includes, at 403, determining a status based at least in part on the signal data. In some embodiments, determining the status can occur periodically, continuously, and/or sporadically during monitoring a patient. The status can correspond to the status of a portion of the patient such as the cardiac system, pulmonary/respiratory system, and/or the like. In some embodiments, determining a status can further be based on data from additional sources (e.g., additional sensors and/or one more ex vivo data sources). Determining cardiac status can include, for example, comparing and synchronizing the signal data and/or artifacts in the signal data to determine the status or functioning of the portions of the patient P. For example, when signal data associated with a first characteristics (e.g., first signal data, electrical signal data) is associated with electrical signals and the signal data associated with the second characteristic (e.g., second signal data, mechanical signal data) is associated with mechanical signals, determining status can include synchronizing an electrical signal curve (from the first signal data) and a mechanical signal curve (from the second signal data) to correlate the signal data. More specifically, determining status can include correlating a cardiac electrical curve with a hemodynamic curve and/or the cardiac electrical curve with a respiratory curve. As another example, determining the status can include determining if features seen in the electrical signal data have an expected corresponding result in the mechanical signal data. As such, using data associated with more than one characteristic provides more sensitivity and specificity in determining cardiac status than when determining cardiac status using just one source of data (e.g., electrical signals alone). Determining a status associated with a cardiac system and a respiratory system is described in further detail in reference to FIGS. 8-11.

At 404, the method 400 optionally includes refining the status using a machine learning model based on the signal data. Refining can include, for example, improving, tuning, updating, and/or verifying the status determined at 403. The machine learning model can be trained based on status associated with data from the at least one sensor. For example, the machine learning model can be trained based on status associated with electrical signal data and mechanical signal data from the patient, historical data, data from studies, medical professional input, and/or the like. In some embodiments, the machine learning model can be trained based on additional patient data (e.g., activity level, height, weight, age, and/or the like). Training on patient data allows for the machine learning model to gain further insight on the patient that may affect the characteristics of the patient measured by the sensors.

In some embodiments, the status may be changed, updated, tuned, etc., based at least in part on comparing the cardiac status from step 403 to more closely align with empirical data, a status determination by one or more trained cardiologists, updated clinical evidence, and/or any suitable known or accepted ground truth. In some embodiments, the machine learning model may be configured to recognize long-term changes in status and/or the signal data and adapt to these changes. For example, the machine learning model may recognize that the mechanical signal data has been decreasing over weeks, months, or years. Recognizing changes allows the machine learning model to adapt the sensitivity along with the patient as the patient changes. In some embodiments, the machine learning model may receive feedback data and/or training sets that are used to further train the machine learning model.

The method 400 includes, at 405, determining, based on the status, if a health event is occurring. Determining if a health event is occurring can include, in some embodiments, comparing the status to an expected status, comparing status to previously gathered status to determine trends, and comparing the outputs of sensors to confirm a health event. For example, if the status indicates that a characteristic is higher than it expected, the presence of a health event may be determined. In some embodiments, a machine learning model can be used to determine if a health event is present based at least in part on the status. For example, the machine learning model can recognize changes in the patient's activity and/or characteristics that can indicate the presence of a health event, or if no health event is occurring. In some embodiments, an indication that a health event is occurring may be sent to the user. In some embodiments, the machine learning model can recognize when the patient's activity is expected and/or predicted to increase. For example, when the patient is exercising, experiencing stress or anxiety, and/or the like. For example, if the sensors include an accelerometer, a determination that the patient's activity is greater than expected can be made when an acceleration value, acceleration vector, change in an acceleration vector, and/or the like indicates that the patient is active, was active, or will be active.

The method 400 includes, at 406, determining if a health event is detected. At 406, the method 400 includes a decision of whether a health event has been detected. If a health event has not been detected, the method 400 returns to step 401 to continue monitoring the heart of the patient. If a health event has been detected, the method 400 continues to step 407.

The method 400 optionally includes, at 407, determining the type of health event. In some embodiments, determining a type of health event can include comparing and matching the status to known health events or previous status. In some embodiments, a machine learning model may be used to match the status or changes in status to a health event. In some embodiments, the type of health event can be sent to a user device and/or the like for review by the user. For example, the type of health event can be reviewed by the patient, a medical proxy, a medical professional, the implanted device, and/or the like. In some embodiments, the type of health event may include a severity score. The severity score can indicate an urgency and/or a health danger associated with the health event. The method 400 includes, at 408, generating a notification indicating a health event is detected. In some embodiments, the notification is generated in response to the health event having a severity score that indicates an urgent health event. In addition to an indication that a health event is occurring, the notification can include additional information about the health event, such as the type of health event, the characteristics associated with the health event, and/or the like. The notification can be sent to an external device, such as a user device, a healthcare provider, an emergency responder, and/or the like. In some embodiments, the notification can be displayed on a wearable. In some embodiments, an indication of the notification can be displayed on the wearable and further information can be displayed on the user device.

While the method 400 is described above as generating a notification associated with a positive detection of a health event, the method 400 may include generating notifications associated with any suitable data and/or determination in addition to and/or as an alternative to the generation of the notification indicating the health event is detected (at 408). For example, such notifications may be indicative of any determination, inference, calculation, and/or monitoring of data received from any number of sensors. Similarly stated, the generation of one or more notifications need not be limited to the determination of a given health event. Rather, the method 400 may include generating a notification indicating any suitable health event, status, characteristic, determination, inference, trend, correlation, recommended treatment regime, etc. (or one or more changes associated therewith).

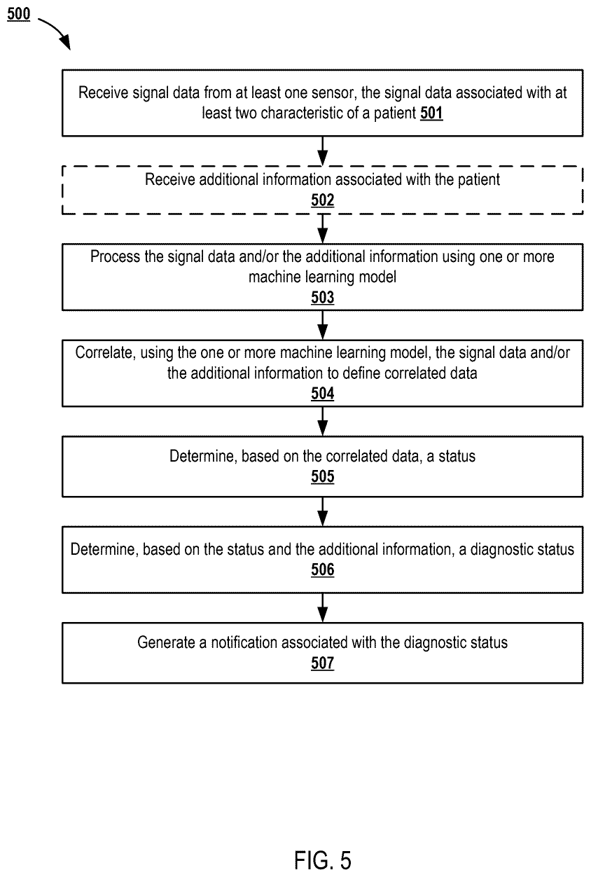

FIG. 5 is a flow chart depicting a method 500 for determining a diagnostic status of a patient, according to an embodiment. In some embodiments, the method 500 can be executed and/or performed by a diagnostic/monitoring system that is functionally and/or structurally similar to the diagnostic/monitoring system 300 of FIG. 3. In some embodiments, the method 500 can be executed by one or more devices. For example, the method 400 can be executed by the sensing device(s) 310 and/or the compute device 330. In some embodiments, the method 500 is configured to use at least two signals form at least two data source(s) (e.g., sensors) to determine a diagnostic status of a patient. For example, the at last two signals can include a cardiac electrical signal and a pressure signal associated with the anterior mediastinum. Using at least two signals reduces the likelihood that a diagnostic status is a false positive. The method 500 includes generating a notification indicating the diagnostic status, which allows for a patient or a user to review the health event and take corrective action. The method 500 can be used in a diagnostic environment which can include an environment where health issues and diagnoses are desired such as in a clinical setting, a hospital, a remote ICU, and/or the like. In some embodiments, the method 500 can be used for monitoring a patient to determine one or more anomaly associated with a patient. For example, the method 500 may be executed and/or performed continuously, periodically, and/or sporadically via a diagnostic/monitoring system to monitor changes to the health of a patient.

The method 500 includes, at 501, receiving signal data from more than one sensor (e.g., functionally and/or structurally similar to the sensors 320). The signal data is associated with at least one characteristic of the patient. In some embodiments, the at least one sensor can include any number of sensors. In some embodiments, the at least two characteristics can include a first characteristic and a second characteristic. In some embodiments, the signal data can include at least one of cardiac electrical data and/or pressure data associated with the anterior mediastinum. In some embodiments, both the signal data is measured in vivo and/or ex vivo. For example, the signal data can be measured in the anterior mediastinum, at the heart wall, or externally on the body of the patient. The first characteristic can be associated with a status and the second characteristic can be associated with the same or different status. In some instances, the characteristics and/or the status(es) can be associated with physiological characteristic(s) and/or status(es), pathophysiological characteristic(s) and/or status(es), and/or combinations thereof. For example, the at least two characteristics can include an electrical characteristic associated with a status and a mechanical characteristic associated with the status. As another examples, the at least two characteristics can include a characteristic (electrical, mechanical, etc.) associated with a first status and a characteristic (electrical, mechanical, etc.) associated with a second status different from the first status. In some embodiments, step 501 may include receiving additional signal data from any number of additional sensors and/or additional data sources (e.g., one or more ex vivo data sources). In some embodiments, step 501 may include receiving the signal data from an external data source (e.g., patient database, etc.). At 502, the method 500 may optionally include receiving additional information associated with the patient. The additional information can include information associated with the patient such as patient age, height, weight, demographic information, medical history, and/or the like.

The method 500 includes, at 503, processing the signal data and/or the additional information using one or more machine learning model. Processing can include using the one or more machine learning model to refine the signal data and/or the additional information. In some embodiments, the machine learning model can be configured to filter out or recognize activity (e.g., breathes, heartbeats, physical activity, etc.) that were determined or missed during an initial determination. In some embodiments, signal data associated with different characteristics and/or sensor can be processed by different machine learning models. For example, a signal data associated with a first characteristic can be processed by a first machine learning model and signal data associated with a second characteristic can be processed by a second machine learning model. In some embodiment, a machine learning model can be associated with multiple types and/or sources of signal data. In some embodiments, the additional information can be associated with a machine learning model that is configured to process the information.

In some embodiments, the one or more machine learning models can be configured to cluster signal data and the additional information based on relevance to a known status or previously collected status. For example, the machine learning models can be configured to cluster signals that are relevant to a status or multiple statuses that the diagnostic/monitoring system is configured to determine. Clustering signals can allow for later correlation (e.g., at 504) of the signals that are relevant to the desired status(es) and further determination of trending of status. For example, if a user indicates that only certain status determinations are desired, clustering may cluster only the signal data and the additional information that is relevant to the status indicated as desired. In some embodiments, processing the signal data can include filtering and/or amplification. In some embodiments, processing the signal data can include generator one or more curve as described in reference to FIGS. 6-7.

The method 500 optionally includes, at 504, correlating, using the one or more machine learning model, the signal data and/or the additional information to define correlated data. In some embodiments, the machine learning model can correlate the signal data and/or the additional information to determine a pattern. In some embodiments, the one or more machine learning models can learn from the outputs of the at least one sensor to determine long-term correlation and changes in correlation between the outputs of the at least one sensor. In some embodiments, the machine learning model used to correlate the signal data and/or the additional informant may be different than the machine learning model(s) used to initially process the output from the signal and/or the additional information. In some embodiments, the machine learning model used to aggregate the outputs from the sensors may be different than the machine learning model(s) used to process the signal data and/or the additional information at 503. In some embodiments, correlating can include correlating the curves generated in step 503. For example, the correlating can include correlating a cardiac electrical curve with a cardiac hemodynamic curve and/or a respiratory pressure curve.

The method 500 includes, at 505, determining, based on the correlated data, a status. In some embodiments, determining the status can utilize one machine learning model. In some embodiments, determining the status can utilize more than one machine learning model. The status can correspond to the status of a portion of the patient such as the cardiac system, hemodynamics, and/or pulmonary/respiratory system. In some embodiments, determining a status can further be based on data from additional sources (e.g., additional sensors and/or one more ex vivo data sources). Determining cardiac status can include, for example, comparing and synchronizing the correlated data and artifacts in the signal data to determine the status or functioning of the portions of the patient. As another example, determining the status can include determining if features seen in the electrical signal data have an expected corresponding result in the mechanical signal data. As such, using data associated with more than one characteristic provides more sensitivity and specificity in determining cardiac status than when determining cardiac status using just one source of data (e.g., electrical signals alone). In some embodiments, determining the status can include determining a status based on the correlated data over a period of time (e.g., in a monitoring context). For example, the status can include changes in the characteristics over a period of time that may be indicative of a disease and/or condition. For example, changes in right atrial pressure, pulmonary capillary wedge pressure, and/or stroke volume (e.g., as determined from the pressure within the anterior mediastinum) can be used to determine that symptoms of heart failure are present.

The method 500 includes, at 506, determining, based on the status and the additional information, a diagnostic status. Determining the diagnostic status can include matching the status with a known diagnosis, or lack thereof. For example, a machine learning model can be used to determine if the status is associated with a known diagnosis. The method 500 includes, at 507, generating a notification associated with the diagnostic status. The notification can be used to indicate to a user of the diagnostic status for monitoring purposes and/or treatment purposes. The notification can be used to indicate to a user that the diagnostic status is ready for review. In some embodiments, the notification can be configured for sending to an emergency responder, a medical provider, secondary user (e.g., other than the patient P), and/or the like. The notification can be used so that corrective action can be taken if necessary.

While the method 500 is described above as generating a notification associated with a positive detection or determination of a diagnostic status, the method 500 may include generating notifications associated with any suitable data and/or determination in addition to and/or as an alternative to the generation of the notification associated with the diagnostic status (at 507). For example, such notifications may be indicative of any determination, inference, calculation, and/or monitoring of data received from any number of sensors. Similarly stated, the generation of one or more notifications need not be limited to the determination of a given diagnostic status. Rather, the method 500 may include generating a notification indicating any suitable health event, status, characteristic, determination, inference, trend, correlation, etc. (or one or more changes associated therewith).

While the method 400 and/or 500 are described above as determining the occurrence of a health event and/or a diagnostic status for a patient, in some implementations, the determination of the health event and/or diagnostic status need not be determined after receiving data from the one or more sensors and/or data sources. For example, in some implementations, data received from the one or more sensors and/or data sources may be stored for a period of time prior to being used in the determination of the health event and/or diagnostic status. In some implementations, the data may be stored locally (e.g., in a memory of the diagnostic device) and then sent to an external device for processing and/or analysis in addition to and/or as an alternative to the determination of the health event and/or diagnostic status. In some implementations, the determination of the health event and/or diagnostic status is based on data received from one or more sensors and/or data sources over a period of time (e.g., an extended period of monitoring and/or the like) during which the diagnostic device can store the data received prior to analyzing. In some implementations, the determination of the health event and/or diagnostic status can be retrospective based on an analysis and/or processing of data received over a period of time and stored, for example, in a memory of the diagnostic device. In some implementations, such a retrospective analysis can be performed on data associated with and/or otherwise from any number of diagnostic devices (e.g., from the diagnostic devices of multiple patients).

Embodiments described herein can include and/or can be used with any suitable amplification device, filtering device, signal processing device, and/or the like to process (or pre-process) data received from one or more sensors. For example, one or more amplifier/filters can be operably coupled between a sensing device such as the sensing device 310 and one or more sensors such as the sensors 320. As described above, the sensing device 1010 can be an implantable medical device configured to sense, detect, monitor, and/or measure one or more bio-signals and/or characteristics of the patient and to use data associated with the bio-signals and/or characteristics to provide diagnostic functionality and/or predictions and/or provide therapeutic/treatment decision-making, which in turn, can be carried out and/or delivered by one or more portions of the device.

At least one amplifier/filter can be included in and/or coupled to the sensing device. A sensor or multiple sensors may couple to the at least one amplifier/filter. The amplifier/filter may be a single amplifier/filter, a cascade or multitude of amplifier/filters, a combination of an amplifier and a filter, multiple combinations of amplifiers and filters, a digital amplification and filtering process performed by a processor, and/or the like. In some embodiments, the amplifier/filter can be and/or can include one or more high-pass or low pass filters that can be configured to filter the pressure data based at least in part on frequency. In some embodiments, the amplifier/filter can be and/or can include any suitable device configured to amplify a magnitude of at least a portion of the data representing pressure changes in the mediastinal space. Such amplification can allow for a greater degree of sensitivity in detecting pressure changes.

In operation, at least one sensor of the sensing device senses, detects, monitors, and/or measures one or more bio-signals which is/are then amplified and filtered, via the amplifier/filter, for use by the sensing device. The signals sensed, detected, monitored, and/or measured by the sensor(s) are fed into the amplifier/filter, which filters, separates, and amplifies one or more portions of the signals. The amplified and filtered/separated signals can represent, for example, pressure curves or other suitable data allowing the sensing device to monitor the health of the patient and/or to diagnose or define a diagnostic prediction associated with the health of the patient (as described above with reference to the system 100 and/or 300).

FIG. 6 is a schematic illustration of a diagnostic/monitoring system 600 (e.g., functionally and/or structurally similar to the diagnostic/monitoring system 300 of FIG. 3) including a sensing device 610 that is configured for use with one or more amplifiers, filters, signal processors, and/or the like. The sensing device 610 can be functionally and/or structurally similar to the sensing device 110 of FIG. 1, the sensing device 210 of FIGS. 2A and 2B, and/or the sensing device(s) 310 of FIG. 3. The sensing device 610 is operatively coupled to a lead 621 that includes, for example, an ECG electrode 624 (configured to detect ECG signals) and a pressure sensor 622 (e.g., configured to detect pressure signals). The sensing device 610 includes a processor 612 (e.g., functionally and/or structurally similar to the processor 312 of FIG. 3), a memory 614 (e.g., functionally and/or structurally similar to the memory 314 of FIG. 3), an I/O device 616 (e.g., functionally and/or structurally similar to the I/O device 316 of FIG. 3) including an antenna 616a, a sensor amplifier 652, an ECG amplifier 654, and an analog-to-digital converter 660. The processor 612, the antenna 616a, and the pressure sensor 622 are coupled electrically to a common power source (e.g., a 3V power source and/or any other suitable power source).

The lead 621 is operatively coupled to the sensing device 610. The lead 621 is configured to deliver and/or place the ECG electrode 624 and the pressure sensor 622 to/in a desired location. In some embodiments, the desired location is remote from the sensing device 610. For example, the lead 621 can be configured to be implanted at least partially within the anterior mediastinum while the sensing device 610 is located outside of the anterior mediastinum. In some embodiments, the lead 621 can have a distal end in the anterior mediastinal (e.g., an end portion including the ECG electrode 624 and/or the pressure sensor 622) and a proximal end outside of the anterior mediastinum (e.g., an end portion coupled to the sensing device 610). In some embodiments, the lead 621 is configured to at least partially engage, contact, and/or be in close proximity to the heart of the patient.

The ECG electrode 624 is configured to measure and/or detect bio-signals associated with an ECG of the heart. In some embodiments, the lead 621 is configured to position the ECG electrode 624 so that it contacts or is maintained in close proximity to the heart to measure and/or detect ECG signals from the heart wall. The lead 621 is configured to position the pressure sensor 622 in the anterior mediastinum (e.g., and not in contact with the heart, lungs, and/or sternum). The pressure sensor 622 is configured to measure and/or detect pressure within the anterior mediastinum.

The sensing device 610 is coupled to the lead 621 such that the signals from the ECG electrode 624 and the pressure sensor 622 are received by the sensing device 610. Specifically, the sensor amplifier 652 is configured to receive the signals from the pressure sensor 622 and the ECG amplifier 654 is configured to receive the signals form the ECG electrode 624. The amplifiers (e.g., the sensor amplifier 652 and the ECG amplifier 654) may be a single amplifier/filter, a cascade or multitude of amplifier/filters, a combination of an amplifier and a filter, multiple combinations of amplifiers and filters, a digital amplification and filtering process(es) or device(s), and/or the like. In some embodiments, the amplifiers can be and/or can include one or more high-pass or low pass filters that can be configured to filter the data based at least in part on frequency or frequency band. In some embodiments, the amplifiers can be and/or can include any suitable device configured to amplify a magnitude of at least a portion of the data representing pressure changes in the mediastinal space and/or ECG changes. Such amplification can allow for a greater degree of sensitivity in detecting pressure changes and/or ECG changes. For example, without amplification, filtering may remove minor changes that may be desirable to be recorded. With amplification, the minor changes also get amplified and thus there is a reduced likelihood of filtering out the minor changes in the signals, thus allowing for a specificity that can allow a user to see desired changes to the signals. In some embodiments, the type of amplification and/or filtering can be associated with the type, size, etc. of changes that are desired to be monitored.

The analog-to-digital (ADC) converter 660 is configured to receive the outputs of the sensor amplifier 652 and the ECG amplifier 654 and convert them from an analog signal to a digital signal. In some embodiments, the ADC converter 660 can be configured to complete one or more of the filtering processes described in reference to the sensor amplifier 652 and/or the ECG amplifier 654. The ADC converter 660 is configured to convert the signals from the amplifiers (e.g., simultaneously in parallel processes) so that the time alignment between the ECG signal and the pressure signal can be maintained. Ensuring time alignment allows for synchronized signals that can be used for real time monitoring purposes. In some embodiments, the ADC converter 660 can have resolution of 8-bits, 16-bits, 32-bits, 64-bits, and/or other higher resolutions. In some embodiments, the ADC converter 660 can be configured to receive signals of varied sample rates. In some embodiments, the sensor amplifier 652, the ECG amplifier 654, and the ADC converter 660 can be and/or can form at least a part of a sensor interface similar to the sensor interface 318 in FIG. 3.

The processor 612 and the memory 614 can be configured to function similar to the processor 312 and the memory 314 described in reference to FIG. 3. The processor 612 receives the converted signals from the ADC converter 660. The converted signals are associated with signals from the ECG electrode 624 and the pressure sensor 622. The processor 612 is configured to execute instructions stored on the memory 614. As described in reference to FIG. 3, the instructions can include various processes associated with monitoring the signals to determine a health status, whether a health event is occurring, a diagnostic status, an anomaly, one or more trends, and/or the like.

The I/O device 616 is configured to receive processed data and/or information from the processor 612 and to send information to the processor 612, as described in reference to FIG. 3. The antenna 616a can be used for communicating with external devices. For example, the antenna 616a can be used to send signals to an external device indicating that a health event is occurring.

FIG. 7 is a schematic illustration of a filtering process 700, according to an embodiment. The filtering process can be executed by a sensing system (e.g., functionally and/or structurally similar to the diagnostic/monitoring system 100 (FIG. 1), 200 (FIGS. 2A and 2B), 300 (FIG. 3), and/or 600 (FIG. 6). For example, in some implementations, the filtering process 700 is a process performed and/or executed by the sensing device 310 of FIG. 3 and/or the sensing device 610 of FIG. 6. In some implementations, the filtering process 700 is a process performed and/or executed by or at a remote compute device (e.g., the compute device 330) and/or in a cloud environment (e.g., a virtual machine or instance executed by hardware). In some implementations, the filtering process 700 can include one or more processes performed and/or executed by a sensing device (e.g., the sensing device 310 and/or 610), and one or more processes performed and/or executed by a remote device (e.g., the remote compute device 330 and/or the like).

The filtering process 700 includes generating a plurality of curves based on a pressure signal 722 and a cardiac electrical signal 724 received from one or more sensors, electrodes, etc. For example, the pressure signal 722 is measured and/or detected by a pressure sensor within the anterior mediastinum. The cardiac electrical signal 724 is measured and/or detected by an ECG electrode that is configured to engage the heart or externally measure the ECG signal. In some embodiments, the pressure signal 722 and the cardiac electrical signal 724 can be signals that are continuously provided to the filtering process 700 so that the data can be processed in real-time or near real-time.

The pressure signal 722 and the cardiac electrical signal 724 are passed through one or more amplifier filter 752, 754. The amplifier filter 752, 754 (e.g., functionally and/or structurally similar to the sensor amplifier 652 and the ECG amplifier 654 of FIG. 6, respectively) are configured to amplify and/or filter the signals as described above in reference to FIG. 6. In some embodiments, the amplification and/or filtering, performed by the amplifier filter 752 can be based at least in part on differing frequencies within the pressure data. For example, pressure changes due to respiration can have a relatively low frequency, while pressure changes due to cardiac activity (e.g., the beating of the heart) can have a relatively have frequency. These frequencies, for example, correspond to and/or are indicative of the underlying physiological processes producing the pressure changes. For example, a normal respiration rate for an adult patient at rest can be around 12-20 breaths per minute, while a normal heart rate for an adult patient at rest can be around 60-100 beats per minute. Thus, pressure changes associated with cardiac function can have a frequency about 5 times faster than a frequency associated with respiratory function. In some instances, pressure changes associated with the basic mediastinal pressure (or background pressure) can take place over an extended period and thus, a frequency of changes to the basic/background mediastinal pressure can have a very low frequency compared to the frequencies associated with respiration and/or cardiac function (e.g., expected to remain substantially consistent/constant for an extended period).

The pressure signals 722 are separated by the amplifier filter 752 into distinguishable and/or distinct curves corresponding to one or more frequency band. The curves include a cardiac hemodynamic curve 762, a respiratory function curve 764, and an anterior mediastinum pressure curve 766. The pressure curves can be further digitally filtered to obtain more stable and precise pressure curves. The cardiac hemodynamic curve 762 represents a portion of the pressure signals 722 in a frequency band associated with the hemodynamic function of the heart. For example, the cardiac hemodynamic curve 762 can be associated pressure changes in the anterior mediastinum as a result of changes in the heart's volume (e.g., systole and diastole). The respiratory function curve 764 represents a portion of the pressure signals 722 in a frequency band associated with the function of the respiratory system. For example, the respiratory function curve 764 can be associated with pressure changes in the anterior mediastinum and/or pleural cavity as a result of changes in lung volume during respiration. The anterior mediastinum pressure curve 766 represents a portion of the pressure signals 722 in a frequency band associated with the background or baseline pressure in the anterior mediastinum. In general, the anterior mediastinum pressure curve 766 for a patient at rest will remain substantially rhythmic, based on the changes from pressure changes from the heart's volume changes and changes in the lung's volume changes. In contrast, abrupt changes in the anterior mediastinum pressure curve 766 would suggest, for example, a significant injury such as a broken rib or puncture of the pleural cavity, while changes over an extended period would suggest a gradual change in physiological or pathophysiological characteristics (e.g., a gradual increase in the size of the heart due to worsening heart disease). In some embodiments, the cardiac electrical signal 724 can be used, after being passed through the amplifier filter 752, to generate cardiac electrical data 768. In some implementations, the cardiac electrical signal 724 can also be used to correlate, corroborate, verify, and/or confirm the amplified and filtered pressure signal 722 to generate the cardiac hemodynamic curve 762. In some embodiments, the amplifier filters 752, 754 can include one or more amplifier/filters configured to amplify and/or filter the electrical signals.

The relationship between the waveform morphology of the cardiac pressure curve and simultaneously recorded waveforms of the cardiac electrical signals can reflect many changes in cardiac function and hemodynamic parameters. The respiratory pressure curve can reflect respiratory rate and rhythm changes, respiratory movement, and respiratory function. The anterior mediastinum pressure curve can reflect long-term, slow mediastinal pressure changes, fluid status, chronic cardiac function changes, heart failure decompensation for both low ejection fraction and preserved ejection fraction patients, and mediastinal diseases. These manifest, for example, in peripheral edema, pulmonary edema, dyspnea, and/or fatigue. Changes in these parameters can then be associated with clinical symptoms of the aforementioned diseases, disorders, or conditions and occur before the onset of symptoms, thereby predicting the onset of symptoms.

In some implementations, data associated with and/or indicative of the cardiac hemodynamic curve 762, the respiratory function curve 764, and the anterior mediastinum pressure curve 766, and data associated with and/or indicative of the cardiac electrical data 768 are all fed to a compute device such as, for example, the processor 612 of the sensing device 610 and at least temporarily stored in the memory 614. In some instances, the sensing device 610 can be programed to monitor the received data and/or to make diagnostic predictions based on the received data. In some instances, the sensing device 610 can be configured to define and/or output a notification (or a signal causing a notification) when a criterion associated with the data is met. For example, as described above with reference the sensing device 310, a notification can be generated when one of the data curves is outside of an expected and/or predefined range. Further analytics as discussed above with respect to any of the systems 100 and/or 300 can be performed. For example, the processor 612 may be able to diagnose, predict, and/or provide treatment for the physiological status of a patient from an algorithmic application stored, for example, within the memory 614. Alternatively or in addition, one or more machine learning models may be used for diagnostic and/or predictive purposes. Moreover, this diagnostic function can be implemented therapeutically. For example, by determining the status and monitoring the changes of respiratory rate, rhythm changes, respiratory movement, respiratory function, pressure changes (e.g., right arterial pressure (RAP), pulmonary capillary wedge pressure (PCWP), central venous pressure (CVP), and/or the like), fluid status, chronic cardiac function changes, stroke volume changes, low ejection fraction, preserved ejection fraction, and mediastinal diseases, the data can influence the timing of operation of an ICD or other medical device designed to treat the heart or other organs of the patient, or pharmacological changes to address status changes. This can allow for improved monitoring (and/or improved specificity of the collected or measured data), which in turn, can reduce the likelihood of false positive diagnoses as well as false positives in therapeutic/treatment decision-making, as described in further detail herein.

Referring generally to FIGS. 8-11, methods for determining cardiac and pulmonary parameters are described. In some embodiments, the methods 800, 1000 can be executed by a system such as the system 100 of FIG. 1, the system 300 of FIG. 3, and/or the system 600 of FIG. 6. Moreover, the methods 800, 1000 can be implemented in an implantable treatment and/or diagnostic device, can be implemented remotely (e.g., at a remote compute device, in a cloud environment, and/or the like), or any suitable combination thereof. When desired, the methods 800, 1000 can be implemented simultaneously to determine both cardiac and pulmonary parameters. The methods 800, 1000 can be used to monitor a patient's health to determine a cardiac status and/or a pulmonary status. Monitoring can aid in determining if a health event is occurring instantaneously (e.g., heart attack, etc.) or if a long-term problem is occurring based on observed changes to cardiac and/or pulmonary parameters.

FIG. 8 is a flow chart depicting a method 800 for determining at least one cardiac parameter, according to an embodiment. Determining the at least one cardiac parameter can be for monitoring, diagnostic, and/or similar reasons. The method 800 can be implemented together with or sequentially with the method 1000. At 801, the method 800 includes engaging at least one electrode with a patient, the at least one electrode configured to measure a cardiac electrical signal. In some embodiments, the cardiac electrical signal is an ECG signal. In some embodiments, the at least one electrode is configured to engage, contact, and/or be in close proximity to a heart of the patient for measuring the cardiac electrical signal extracardially. In some embodiments, the at least one electrode can be delivered to the anterior mediastinal space via a lead. At 802, the method 800 includes disposing at least one pressure sensor within an anterior mediastinal space of a patient. In some embodiments, the pressure sensor is delivered to the anterior mediastinal space via a lead. In some embodiments, the pressure sensor and the at least one electrode are delivered to the anterior mediastinum in tandem (e.g., via the same lead).

At 803, the method 800 optionally includes amplifying and filtering measurements received from the at least one pressure sensor and the at least one electrode to define processed pressure data and processed electrode data. In some embodiments, amplifying and filtering may be by a single amplifier/filter, a cascade or multitude of amplifier/filters, a combination of an amplifier and a filter, multiple combinations of amplifiers and filters, a digital amplification and filtering process, and/or the like. In some embodiments, the amplifiers can be and/or can include one or more high-pass or low pass filters that can be configured to filter the data based at least in part on frequency. In some embodiments, the amplifiers can be and/or can include any suitable device configured to amplify a magnitude of at least a portion of the data representing pressure changes in the mediastinal space and/or ECG changes. Such amplification can allow for a greater degree of sensitivity in detecting pressure changes and/or ECG changes.

At 804, the method 800 includes generating a cardiac and/or hemodynamic curve based on the processed pressure data. In some embodiments, the cardiac curve is as cardiac hemodynamic curve. In some embodiments, the cardiac hemodynamic curve can be associated with the change in the heart's stroke volume during the operation of the heart. It can also be associated with various pressures in the circulatory system, including right arterial pressure, central venous pressure, pulmonary arterial pressure, and/or pulmonary capillary wedge pressure, left ventricle end of diastole pressure, and others. For example, the cardiac curve can be associated with the systolic and diastolic movement of the heart within the thoracic cavity. In some embodiments, generating the cardiac curve is additionally based on the processed electrode data. For example, the electrode data can be correlated with the pressure data to generate the cardiac curve.

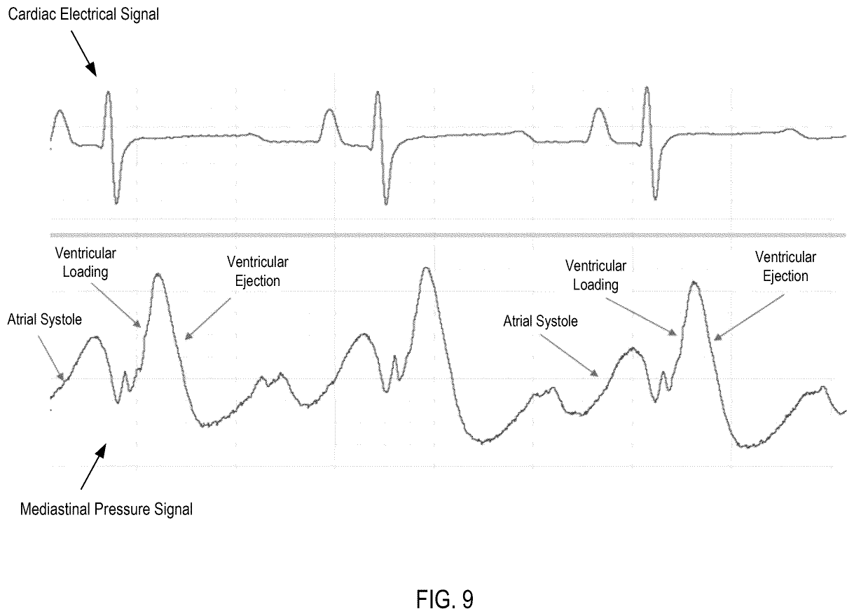

At 805, the method 800 includes determining at least one cardiac parameter based on at least one of the cardiac curve or the processed electrode data. The at least one cardiac parameter can be a cardiac parameter associated with the function of the heart that can be determined based on the cardiac curve and/or the processed electrode data. For example, the at least one cardiac parameter can include changes in heart volume, systolic movements (e.g., systolic function), diastolic movements (e.g., diastolic function), cardiac stroke volume, rate of cardiac contraction, rate of cardiac relaxation, and/or other cardiac parameters that can be determined based on changes in a heart's volume. Additionally, the at least one cardiac curve and the processed electrode data can be correlated to determine excitation-contraction coupling time and atrial-ventricular preload and afterload. Atrial-ventricular preload and afterload can be determined based on a time difference between the peaks of an ECG waveform and a cardiac hemodynamic curve as seen in FIG. 9. The preload of the atria and ventricles includes volume load while the afterload includes pressure load. In some embodiments, central venous pressure can be calculated based on a baseline pressure associated with a substernal space.

At 806, the method 800 optionally includes assessing a cardiac status of the patient based at least in part on the at least one cardiac parameter. The cardiac status can be assessed in a short-term or a long-term frame. For example, in the short-term frame, the cardiac status can be assessed for any sudden health events, anomalies, or diagnoses such as a heart attack. Long-term frame can include gradual changes to the overall cardiac function of the heart, including changes in the hemodynamic output, blood pressure, heart rate, functional arterial pressures, functional venous pressures, and/or the like. The long-term frame can uncover gradual changes that may not be apparent over the shorter period of time. If an anomaly, health event, and/or a diagnosis is made or detected, a notification can be generated for sending to a user, as described herein.

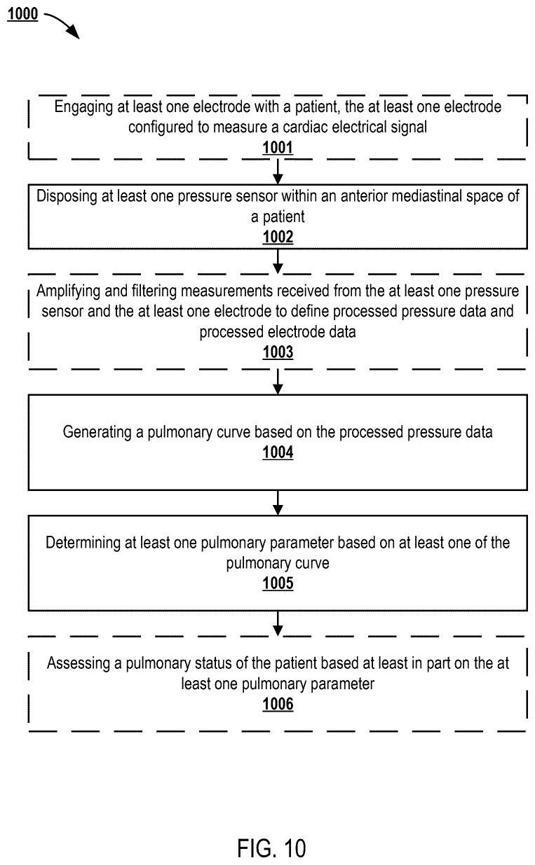

FIG. 10 is a flow chart depicting a method 1000 for determining at least one pulmonary parameter, according to an embodiment. Determining the at least one pulmonary parameter can be for monitoring, diagnostic, and/or similar reasons. The method 1000 can be implemented together with or sequentially with the method 800. At 1001, the method 1000 optionally includes engaging at least one electrode with a patient, the at least one electrode configured to measure a cardiac electrical signal. In some embodiments, the cardiac electrical signal is an ECG signal. In some embodiments, the at least one electrode is configured to engage a heart of the patient for measuring the cardiac electrical signal intracardially. In some embodiments, the at least one electrode can be delivered to the anterior mediastinal space via a lead. In some embodiments, 1001 can be optional when only pulmonary characteristics are desired. At 1002, the method 1000 includes disposing at least one pressure sensor within an anterior mediastinal space of a patient. In some embodiments, the pressure sensor is delivered to the anterior mediastinal space via a lead. In some embodiments, the pressure sensor and the at least one electrode are delivered to the anterior mediastinum in tandem (e.g., via the lead).

At 1003, the method 1000 optionally includes amplifying and filtering measurements received from the at last one pressure sensor and the at least one electrode to define processed pressure data and processed electrode data. In some embodiments, amplifying and filtering may be by a single amplifier/filter, a cascade or multitude of amplifier/filters, a combination of an amplifier and a filter, multiple combinations of amplifiers and filters, a digital amplification and filtering process, and/or the like. In some embodiments, the amplifiers can be and/or can include one or more high-pass or low pass filters that can be configured to filter the data based at least in part on frequency. In some embodiments, the amplifiers can be and/or can include any suitable device configured to amplify a magnitude of at least a portion of the data representing pressure changes in the mediastinal space and/or ECG changes. Such amplification can allow for a greater degree of sensitivity in detecting pressure changes and/or ECG changes.

At 1004, the method 1000 includes generating a pulmonary curve based on the processed pressure data. The pulmonary curve is a curve associated with the function of the respiratory cycle. The pulmonary curve corresponding to the contraction and the relaxation of the respiratory muscles and diaphragm that cause the shape and volume of the thoracic cavity to change with each breath which results in changes in pressure within the anterior mediastinum.

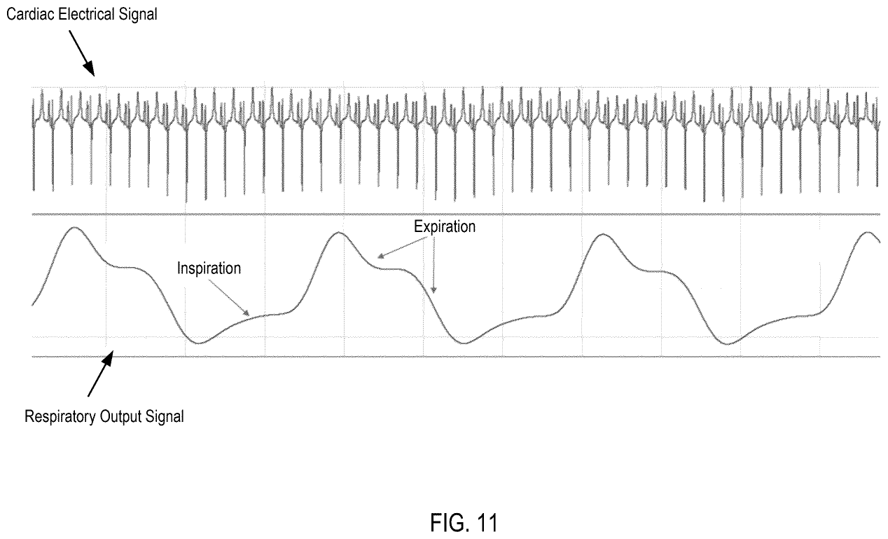

At 1005, the method 1000 includes determining at least one pulmonary parameter based on at least one of the pulmonary curve or the processed electrode data. The at least one cardiac parameter can be a pulmonary parameter associated with the function of the respiratory system based on the pulmonary curve. For example, the at least one pulmonary parameter can include changes in lung volume, resistance/rate/rhythm/timing of inspiration/expiration (as seen in FIG. 11, which depicts a cardiac electrical signal (surface ECG) measurement and a filtered pressure signal during sinus rhythm indicative of or corresponding to respiratory output (e.g., a respiratory output signal or pulmonary curve)), and/or the like. In some embodiments, the at least one pulmonary parameter is/are determined based on the area under the curve. For example, the area under the curve can be used to determine a tidal volume, inspiratory capacity, minute ventilation (e.g., sum of tidal volume within a minute). In some embodiments, the slope of the curve during inspiration/expiration can be used to determine inspiration/expiration flow rate, maximum inspiratory pressure, maximum expiration pressure, duration of inspiration, duration of expiration, and/or the like.

At 1006, the method 1000 includes assessing a pulmonary status of the patient based at least in part on the at least one pulmonary parameter. The pulmonary status can be assessed in a short-term or a long-term frame. For example, in the short-term frame, the pulmonary status can be assessed for any sudden health events, anomalies, or diagnoses such as respiratory failure. Long-term frame can include gradual changes to the overall pulmonary function of the lungs, including changes in the lung volume, breathing rate, dyspnea, and habits (e.g., rhythm, time, etc.), and/or the like. The long-term frame can uncover gradual changes that may not be apparent over the shorter period of time. If an anomaly, health event, and/or a diagnosis is made or detected, a notification can be generated for sending to a user, as described herein.

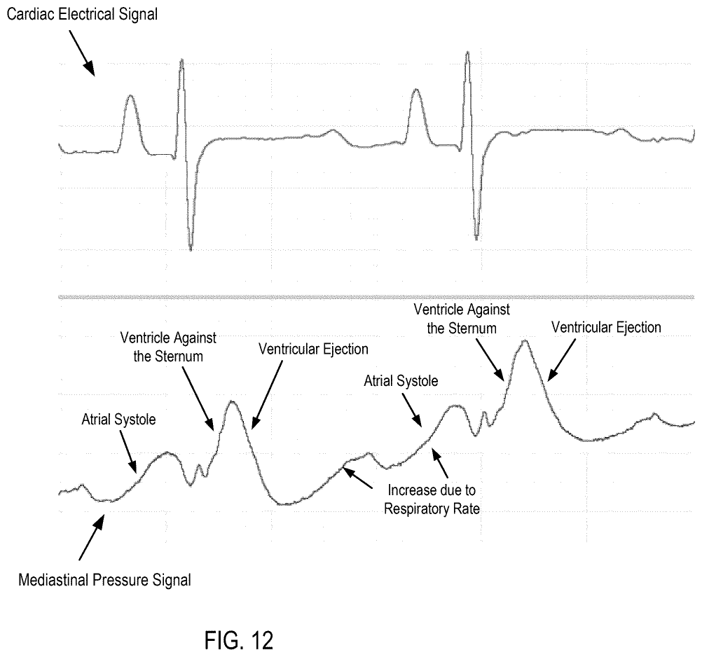

Referring generally to FIGS. 12-15, various comparisons between cardiac electrical signals (e.g., an electrocardiogram (ECG) signals), labeled and mediastinal pressure signals are shown. The pressure signals, labeled “Mediastinal Pressure Signal” is associated with pressure signals measured within the anterior mediastinum and can allow for determining various parameters associated with the function of the heart and/or lungs and can be used for monitoring a patient to determine if a health event is occurring at a given time or if changes to the patient status, health status, condition, pathology, etc. are occurring over time to allow for desired treatment. As seen in FIG. 12, which depicts a cardiac electrical signal at normal sinus rhythm (top) and a corresponding mediastinal pressure signal (bottom). The mediastinal pressure signal is an unfiltered signal that shows a hemodynamic response (and/or pressure changes) due to the movement of the heart during the cardiac cycle. A difference or change in the pressure signal over two heartbeats is shown due to function of the respiratory system. Specifically, the pressure signal during the first heartbeat is generally lower than the pressure signal during the second heartbeat as an inhalation increases the pressure in the anterior mediastinum. In some embodiments, a respiratory signal can be used as a baseline pressure when determining cardiac functions.

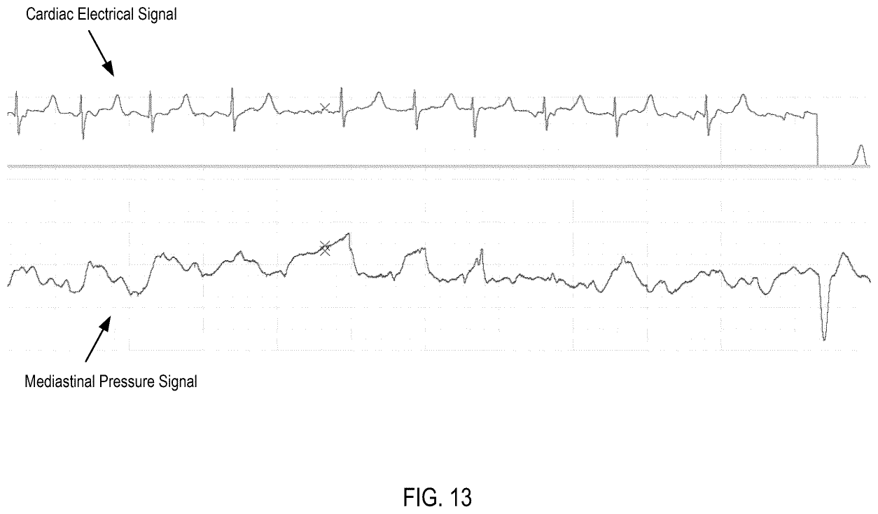

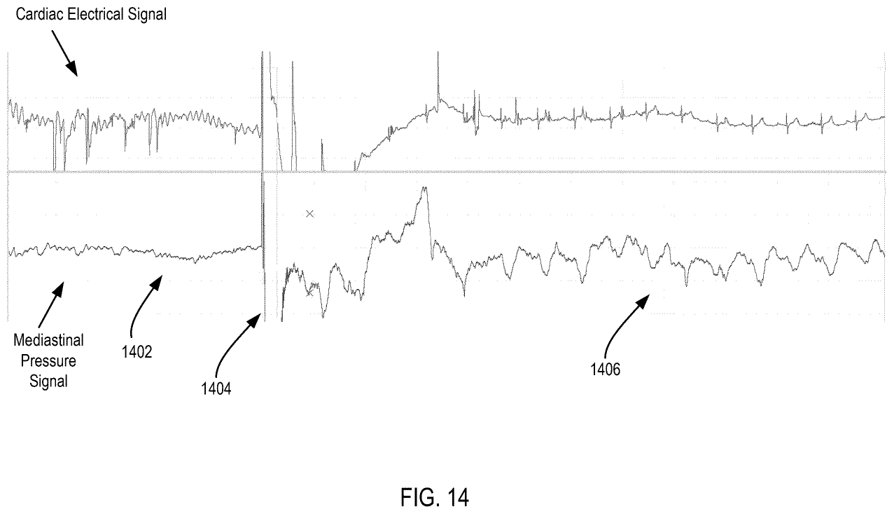

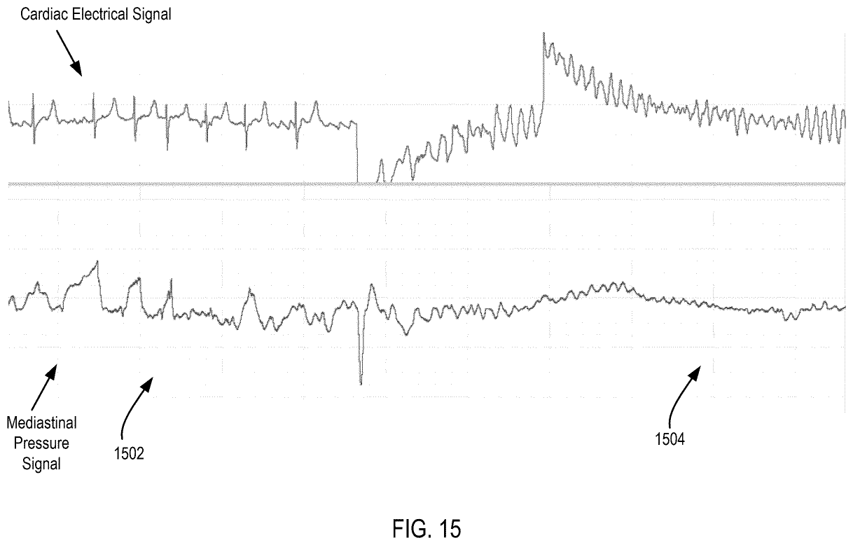

The pressure and ECG signals can be used for determining and/or predicting if various conditions are present. For example, FIG. 13 depicts the cardiac electrical signal (top) and the corresponding mediastinal pressure signal (bottom) during atrial fibrillation. Inclusion, evaluation, and/or correlation of the mediastinal pressure signals (e.g., or at least one or more amplified and filtered portion(s) thereof) can allow for the determination, inference, and/or prediction of a hemodynamic output of the heart, which in turn, can allow for an atrial fibrillation determination based on the ECG signals to be confirmed and/or rejected, thereby reducing the likelihood of a treatment device providing an inappropriate shock. FIG. 14 depicts the cardiac electrical signal (top) and the corresponding mediastinal pressure signals (bottom) during ventricular fibrillation conversion. During conversion, at 1402, ventricular fibrillation can be detected and/or predicted based on the cardiac electrical signal and, for example, confirmed based on the mediastinal pressure signal. The mediastinal pressure signal shows rapid but relatively low amplitude pressure changes, which is associated with and/or indicative of reduced hemodynamic output). In some instances, when ventricular fibrillation is detected and/or confirmed, a defibrillation shock can be delivered as treatment, at 1404. After recovery from the shock treatment, the heart returns to normal sinus rhythm, at 1406. Similar to FIG. 13, the hemodynamic output (e.g., the mediastinal pressure signal or at least one or more amplified and filtered portion(s) thereof) can be used to confirm what is indicated, determined, and/or predicted by the ECG signal. FIG. 15 depicts the cardiac electrical signal (top) and the mediastinal pressure signal (bottom) during atrial fibrillation and ventricular fibrillation. At 1502, the patient is undergoing atrial fibrillation prior to inducing ventricular fibrillation (via a delivered electrical impulse or shock), after which, at 1504, the patient is experiencing ventricular fibrillation. FIG. 15 depicts how the ECG signal and the mediastinal pressure signal (or at least one or more amplified and filtered portion(s) thereof, a hemodynamic output determined or inferred based on the mediastinal pressure signal, and/or the like) are consistent during both atrial fibrillation and during ventricular fibrillation.

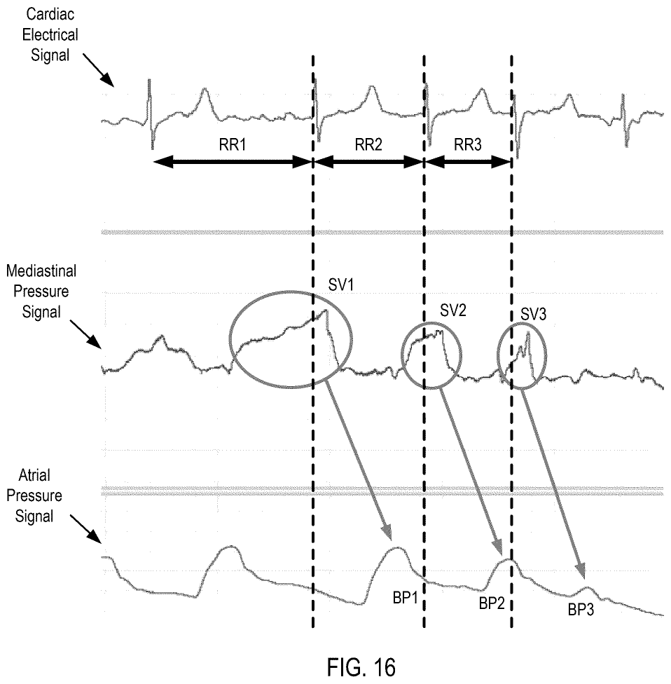

FIG. 16 depicts a comparison between a cardiac electrical signal (e.g., an ECG signal), a mediastinal pressure signal (e.g., a hemodynamic signal), and an arterial pressure signal (e.g., an intra-arterial pressure). As seen in FIG. 16, the R-R intervals (e.g., the time between two R waves on the ECG signal (RR1, RR2, RR3)) are associated with and/or correlate to the stroke volume (SV1, SV2, SV3) shown in the hemodynamic signal. The differences in the R-R intervals lead to differences in the stroke volume (e.g., area under the curve) as the R-R interval differences correspond to differences in ventricular filling. The arterial pressure signals show arterial blood pressure (BP1, BP2, BP3) that is associated with the stroke volumes of the hemodynamic output and thus confirm the differences in the stroke volume. In FIG. 16, the ECG signal indicates that atrial fibrillation is occurring, which results in or is associated with varied R-R intervals. As the R-R interval decreases, the ventricles have less time to fill, as shown by SV2/SV3 and BP2/BP3. In some implementations, these measurements can be used for determining heart failure status and/or worsening heart failure status based on trends in the changes of the stroke volume and blood pressure. In some embodiments, these signals, measurements, and determinations can be performed using an ambulatory diagnostic/treatment device such as any of those described herein.

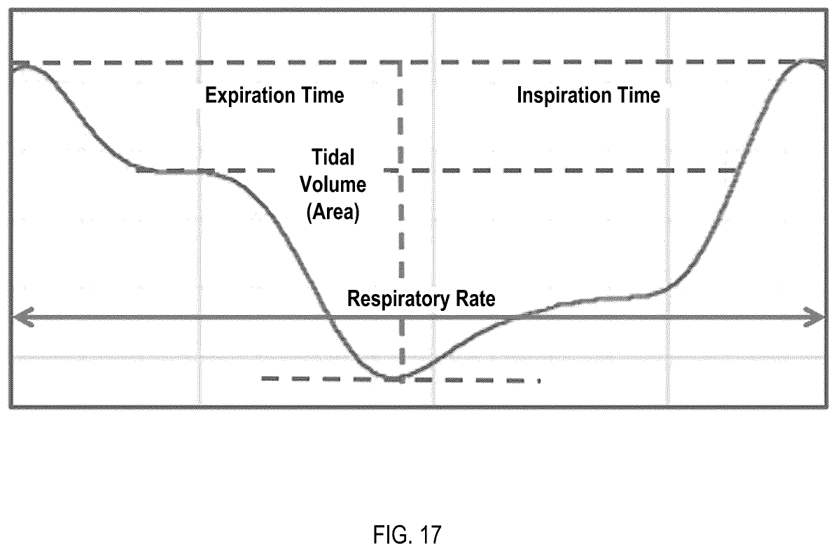

FIG. 17 depicts a filtered and amplified mediastinal pressure signal associated with and/or indicative of a respiratory curve, according to an embodiment. As seen in the respiratory curve, the respiratory rate is associated with a peak-to-peak time, the portion of the curve when the pressure is decreasing to a local minimum is the expiration time, and the portion of the curve from the local minimum to the next peak is the inspiration time. Further, from the respiratory curve, a tidal volume can be determined based on the area within the curve between the peaks. In some embodiments, the volume of air moved in each breath can be determined based on the respiratory curve. The respiratory curve can be used to monitor changes over a period of time in the overall respiratory rate, expiration time, inspiration time, tidal volume, which may indicate a change in the health status of a patient. For example, the changes over time can be indicative of and/or can predict new or worsening shortness of breath, new or worsening pulmonary edema, new or worsening peripheral edema, and/or the like. In some implementations, tracking (e.g., daily, weekly, monthly) of the changes in the patient's status can indicate one or more conditions for which physician intervention is desirable to reduce the likelihood of hospitalization. Said another way, the tracking of the changes in the patient's status can allow for the detection and/or prediction of new or worsening symptoms, conditions, pathologies, etc., allowing for the delivery or adjustment of treatment prior to patient being in an emergency or critical state (e.g., often requiring immediate medical attention, hospitalization, and/or the like).

In some implementations, correlating the pressure signal data and the electrocardiogram signal data can facilitate, improve, and/or confirm conclusions drawn based on the electrocardiogram signal data. For example, in some instances, electrical signals associated with an electrical wave during a specific interval of the cardiac cycle may mimic or may be misidentified as an electrical wave of a different interval of the cardiac cycle. For example, a P wave may be misidentified as a T wave, and/or the like. However, correlating the pressure signal data with the electrocardiogram signal data can confirm, based on detected or inferred hemodynamic status, that the electrocardiogram signal data is associated with the P wave.

In some instances, the correlation of the electrical signal data and the hemodynamic status (e.g., detected, determined, and/or inferred by/from the pressure signal data) can increase a confidence associated with diagnosing or identifying a present health state such as, for example, arrhythmia, fibrillation, and/or the like. The increased confidence in diagnosing or identifying the health state, in turn, can improve treatment decision making such as whether an ICD should provide shock treatment. As an alternative or in addition to improved treatment decision making, monitoring and/or collecting the electrical signal data and the mechanical signal data over a given time can allow a diagnostic system such as those described herein to identify changes in the data that may be indicative of emerging or worsening health conditions such as chronic cardiac conditions like congestive heart failure, heart failure decomposition, heart valve failure or regurgitation, worsening stenosis, etc.; chronic pulmonary conditions like emphysema, COPD, asthma, lung cancer or tumors, etc.; and/or any other suitable health condition. Moreover, based on identifying new or worsening physiological or pathophysiological characteristics, conditions, states, etc., the embodiments and methods herein can provide notification(s) associated with the characteristics, conditions, states, etc. to the patient or a healthcare provider; indication(s), recommendation(s), and/or prescription(s) to change a treatment regimen (e.g., increasing the dosage of a medication or the like) or lifestyles (e.g., eating healthier, increasing a frequency or intensity of one or more exercises, etc.); one or more prediction(s) or the like associated with the characteristics, conditions, states, etc. (e.g., long-term outlook, predicted or projected disease progression), and/or the like.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; solid state storage media such as a solid state drive (SSD) and/or a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a general-purpose processor, an FPGA, an ASIC, and/or the like. Software modules (executed on hardware) can be expressed in a variety of software languages (e.g., computer code), including C, C++, Java™, Ruby, Visual Basic™, Python™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools, and/or combinations thereof (e.g., Python™). Additional examples of computer code include, but are not limited to, control signals, encrypted code, and compressed code.

While various schematics, embodiments, and/or embodiments have been described above, it should be understood that they have been presented by way of example only, and not limitation. Various modifications, changes, and/or variations in form and/or detail may be made without departing from the scope of the disclosure and/or without altering the function and/or advantages thereof unless expressly stated otherwise. Likewise, while embodiments and/or features, components, configurations, aspects, etc. thereof may be described above in the context of certain embodiments, it should be understood that such embodiments are presented by way of example only and not limitation. Any of the embodiments and/or features, components, configurations, aspects, etc. thereof can be used in, and/or adapted for use in, other embodiments unless expressly stated otherwise. Functionally equivalent embodiments, implementations, and/or methods, in addition to those described herein, will be apparent to those skilled in the art from the foregoing descriptions and are intended to fall within the scope of the disclosure.

Where schematics, embodiments, and/or embodiments described above indicate certain components arranged in certain orientations, configurations, or positions, the arrangement of components may be modified. Although various embodiments have been described as having particular features, configurations, and/or combinations of components, other embodiments are possible having a combination of any features, configurations, and/or components from any of embodiments described herein, except mutually exclusive combinations. The embodiments described herein can include various combinations and/or sub-combinations of the functions, components, configurations, and/or features of the different embodiments described.

The specific configurations of the various components can also be varied. For example, the size and specific shape of the various components can be different from the embodiments shown, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise.

Where methods described above indicate certain events occurring in certain order, the ordering of certain events may be modified. Additionally, certain of the events may be performed concurrently in a parallel process, when possible, as well as performed sequentially as described above. While methods have been described as having particular steps and/or combinations of steps, other methods are possible having a combination of any steps from any of methods described herein, except mutually exclusive combinations and/or unless the context clearly states otherwise.

What is claimed is:

1. A system for ambulatory health monitoring, the system comprising:
an electrical sensor configured to be disposed in an anterior mediastinum of a patient, the electrical sensor configured to detect an electrical signal radiating from an external surface of a heart of the patient;
a pressure sensor configured to be disposed in the anterior mediastinum, the pressure sensor configured to detect a pressure signal in the anterior mediastinum; and
a sensing device coupled to the electrical sensor and the pressure sensor, the sensing device including a memory and a processor, the processor configured to execute instructions stored in the memory operable to cause the processor to:
receive electrical signal data from the electrical sensor and pressure signal data from the pressure sensor;
determine, based on the electrical signal data and the pressure signal data and at least one cardiac parameter;
monitor the at least one cardiac parameter over a period of time to determine if a change in the cardiac parameter has occurred; and
responsive to determining that the change in the cardiac parameter has occurred, execute at least one action associated with the change.

2. The system of claim 1, further comprising:
a lead configured to be implanted in the anterior mediastinum of the patient and coupled to the sensing device, wherein the electrical sensor and the pressure sensor are coupled to the lead.

3. The system of claim 2, wherein the sensing device is at least one of a pacemaker, an implantable cardioverter defibrillator (ICD), a cardiac resynchronization therapy defibrillator (CRT-D), or a ventricular assist device (VAD).

4. The system of claim 1, wherein the processor is further configured to execute instructions stored in the memory that cause the processor to:
amplify and filter the pressure signal data to separate the pressure signal data into a plurality of frequency bands;
define a hemodynamic curve corresponding to the pressure signal data in a first frequency band; and
define a pulmonary curve corresponding to the pressure signal data in a second frequency band different from the first frequency band.

5. The system of claim 4, wherein the determining of the at least one cardiac parameter is based on the electrical signal data, the hemodynamic curve, and the pulmonary curve, the monitoring of the at least one cardiac parameter over the period of time includes monitoring a change in each of the electrical signal data, the hemodynamic curve, and the pulmonary curve.

6. The system of claim 4, wherein the processor is further configured to execute instructions stored in the memory that cause the processor to:
define an anterior mediastinal pressure curve corresponding to the pressure signal data in a third frequency band different from the second frequency band and the first frequency band,
wherein the determining of the at least one cardiac parameter is based on the electrical signal data, the hemodynamic curve, the pulmonary curve, and the anterior mediastinal pressure curve, and
wherein the monitoring of the at least one cardiac parameter over the period of time includes monitoring a change in each of the electrical signal data, the hemodynamic curve, the pulmonary curve, and the anterior mediastinal pressure curve.

7. The system of claim 6, wherein the change in the cardiac parameter is associated with at least one of a change in a central venous pressure, a right atrial pressure, a pulmonary capillary wedge pressure, a tidal volume, a respiratory rate, a stroke volume, a fluid load in the anterior mediastinum, or a mediastinal neoplasm.

8. The system of claim 1, wherein the change in the cardiac parameter is indicative of worsening heart failure.

9. A method of using an implanted ambulatory system for health monitoring, the method comprising:
receiving electrical signal data from an electrical sensor disposed in an anterior mediastinum of a patient, the electrical sensor configured to detect electrical signals radiating from an external surface of a heart;
receiving pressure signal data from a pressure sensor disposed in the anterior mediastinum of a patient, the pressure sensor configured to detect a pressure signal in the anterior mediastinum;
determining, based on the electrical signal data and the pressure signal data, at least one cardiac parameter;
monitoring the at least one cardiac parameter over a period of time to determine if a change in the cardiac parameter has occurred; and
executing, in response to determining that the change in the cardiac parameter has occurred, at least one action associated with the change.

10. The method of claim 9, further comprising:
amplifying and filtering the pressure signal data to separate the pressure signal data into a plurality of frequency bands;
defining a hemodynamic curve corresponding to the pressure signal data in a first frequency band; and
defining a pulmonary curve corresponding to the pressure signal data in a second frequency band different from the first frequency band.

11. The method of claim 10, wherein the determining of the at least one cardiac parameter is based on the electrical signal data, the hemodynamic curve, and the pulmonary curve, and
wherein the monitoring of the at least one cardiac parameter over the period of time includes monitoring a change in each of the electrical signal data, the hemodynamic curve, and the pulmonary curve.

12. The method of claim 9, further comprising:
amplifying and filtering the pressure signal data to separate the pressure signal data into a plurality of frequency bands;
defining a hemodynamic curve corresponding to the pressure signal data in a first frequency band;
defining a pulmonary curve corresponding to the pressure signal data in a second frequency band different from the first frequency band; and
defining an anterior mediastinal pressure curve corresponding to the pressure signal data in a third frequency band different from the first frequency band and the second frequency band.

13. The method of claim 12, wherein the determining of the at least one cardiac parameter is based on the electrical signal data, the hemodynamic curve, the pulmonary curve, and the anterior mediastinal pressure curve, and
wherein the monitoring of the at least one cardiac parameter over the period of time includes monitoring a change in each of the electrical signal data, the hemodynamic curve, the pulmonary curve, and the anterior mediastinal pressure curve.

14. The method of claim 13, wherein the change in the cardiac parameter is indicative of at least one of a change in a central venous pressure, right atrial pressure, a pulmonary capillary wedge pressure, a tidal volume, a respiratory rate, a stroke volume, a fluid load, or a mediastinal neoplasm.

15. The system of claim 9, wherein the change in the cardiac parameter is indicative of worsening heart failure.

16. The method of claim 9, wherein the at least one action includes sending a signal representing at least one a notification, a trigger to provide treatment, or an alarm.

17. A non-transitory processor-readable medium storing code representing instructions to be executed by a processor, the code comprising code to cause the processor to:
receive electrical signal data from an electrical sensor disposed in an anterior mediastinum of a patient, the electrical sensor configured to detect electrical signals radiating from an external surface of a heart;
receive pressure signal data from a pressure sensor disposed in the anterior mediastinum of a patient, the pressure sensor configured to detect a pressure signal in the anterior mediastinum;
define a hemodynamic curve based on a first portion of the pressure signal data in a first frequency band and a pulmonary curve based on a second portion of the pressure signal data in a second frequency band different from the first frequency band; and
monitor at least one cardiac parameter over a period of time based at least in part on changes in the electrical signal data, the hemodynamic curve, and the pulmonary curve.

18. The non-transitory processor-readable medium of claim 17, wherein the code further comprising code to cause the processor to:
responsive to determining that a change in the at least one cardiac parameter has occurred, execute at least one action associated with the change.

19. The non-transitory processor-readable medium of claim 18, wherein the at least one action includes sending a signal representing at least one a notification, a trigger to provide treatment, or an alarm.

20. The non-transitory processor-readable medium of claim 17, wherein the code to cause the processor to define the hemodynamic curve and the pulmonary curve further causes the processor to:
amplify and filter the pressure signal data to separate the pressure signal data into at least the first portion of the pressure signal data in the first frequency band and the second portion of the pressure signal data in the second frequency band.

21. The non-transitory processor-readable medium of claim 20, wherein the code to cause the processor to define the hemodynamic curve and the pulmonary curve further causes the processor to:
amplify and filter the pressure signal data to separate the pressure signal data into the first portion of the pressure signal data in the first frequency band, the second portion of the pressure signal data in the second frequency band, and a third portion of the pressure signal data in a third frequency band different from first frequency band and the second frequency band, and
define an anterior mediastinal pressure curve based on the third portion of the pressure signal data in the third frequency band.

22. The non-transitory processor-readable medium of claim 21, wherein the code to cause the processor to monitor the at least one cardiac parameter further causes the processor to:
monitor the at least one cardiac parameter over the period of time based at least in part on changes in the electrical signal data, the hemodynamic curve, the pulmonary curve, and the anterior mediastinal pressure curve.

23. The non-transitory processor-readable medium of claim 22, wherein the at least one cardiac parameter is associated with a chronic cardiac condition.

24. A method of using an implanted ambulatory system for health monitoring, the method comprising:
receiving electrical signal data from an electrical sensor disposed in an anterior mediastinum of a patient, the electrical sensor configured to detect electrical signals radiating from an external surface of a heart;
receiving pressure signal data from a pressure sensor disposed in the anterior mediastinum, the pressure sensor configured to detect pressure signals in the anterior mediastinum;
amplifying and filtering the pressure signal data to define a hemodynamic curve corresponding to pressure signals in a first frequency band and a pulmonary curve corresponding to pressure signals in a second frequency band different from the first frequency band;
defining a change in a health status of the patient over a period of time based on (i) the electrical signal data, the hemodynamic curve, and the pulmonary curve and (ii) a change in at least one of the electrical signal data, the hemodynamic curve, or the pulmonary curve over the period of time; and
executing at least one action associated with the change in the health status.

25. The method of claim 24, wherein the at least one action includes sending a signal representing at least one a notification, a trigger to provide treatment, or an alarm.

26. The method of claim 24, further comprising:
executing a machine learning model to correlate the electrical signal data, the hemodynamic curve, and the pulmonary curve.

27. The method of claim 24, wherein the amplifying and filtering of the pressure signal data further includes defining an anterior mediastinal pressure curve corresponding to pressure signals in a third frequency band different from the first frequency band and the second frequency band.

28. The method of claim 27, wherein the defining of the change in the health status of the patient over the period of time is based on (i) the electrical signal data, the hemodynamic curve, the pulmonary curve, and the anterior mediastinal pressure curve, and (ii) a change in at least one of the electrical signal data, the hemodynamic curve, the pulmonary curve, or the anterior mediastinal pressure curve over the period of time.

29. The method of claim 28, wherein the change in at least one of the electrical signal data, the hemodynamic curve, the pulmonary curve, or the anterior mediastinal pressure curve over the period of time associated with at least one of a change in a central venous pressure, right atrial pressure, a pulmonary capillary wedge pressure, a tidal volume, a respiratory rate, a stroke volume, a fluid load, or a mediastinal neoplasm.

30. The method of claim 29, wherein the health status is a heart failure status.
